(19) United States
(12) Patent Application Publication
(10) Pub. No.: US 2022/0275363 A1
(43) Pub. Date: Sep. 1, 2022

(54) METHODS FOR IDENTIFYING GENOMIC SAFE HARBORS

(71) Applicant: MEMORIAL SLOAN-KETTERING CANCER CENTER, New York, NY (US)

(72) Inventors: Michel Sadelain, New York, NY (US); Ashlesha Odak, Mumbai (IN)

(73) Assignee: MEMORIAL SLOAN-KETTERING CANCER CENTER, New York, NY (US)

(21) Appl. No.: 17/697,028

(22) Filed: Mar. 17, 2022

Related U.S. Application Data
(63) Continuation of application No. PCT/US2020/051253, filed on Sep. 17, 2020.
(60) Provisional application No. 62/901,459, filed on Sep. 17, 2019.

(51) Int. Cl. C12N 15/10; C07K 14/725; C12N 15/90; C12N 9/22

(52) U.S. Cl. CPC C12N 15/1089; C07K 14/7051; C12N 15/907; C12N 9/22; C12N 2750/14143; C12N 2310/20

(57) ABSTRACT

The present disclosure provides methods for identifying genomic safe harbors in a genome (e.g., a human genome).

### CROSS-REFERENCE TO RELATED APPLICATIONS

This application is a continuation of International Application No. PCT/US20/051253, filed Sep. 17, 2020, which claims priority to U.S. Provisional Application No. 62/901,459 filed Sep. 17, 2019, the contents of each of which are incorporated by reference in their entireties herein, and to which each of which priority is claimed.

### 1. TECHNICAL FIELD

The present disclosure provides methods for identifying genomic safe harbors (GSHs) in a genome (e.g., a human genome).

### 2. BACKGROUND

Modification of genomes by the stable insertion of functional transgenes is of great value in biomedical research and medicine. Genetically modified cells are also valuable for the study of gene function, and for creating reporter systems. The reliable function of the introduced transgenes are important for the applications of the genetically modified cells. However, randomly inserted transgenes, i.e., random integration, are subject to position effects and silencing, making their expression unreliable and unpredictable. Reciprocally, newly integrated transgenes may alter the expression of the endogenous genes near the integration site, potentially affecting cell behavior or promoting cellular transformation.

Thus, there remain needs for methods for identifying chromosomal locations where transgenes can integrate and function in a predictable and reliable manner.

### 3. SUMMARY OF THE INVENTION

The present disclosure provides methods for identifying GSHs in a genome (e.g., a human genome).

The present disclosure provides methods for selecting candidates GSHs for targeted integration. In certain embodiments, the method comprises screening a plurality of loci within a genome, evaluating the position of the loci, and identifying a locus as an GSH if such locus is (a) located at a distance of more than about 50 kb from the 5′ end of each gene of the genome; (b) located at a distance of more than about 300 kb from each cancer-related gene of the genome; (c) located outside each gene transcription unit of the genome; locate outside of each ultra-conserved region of the genome; (d) located outside of each non-coding RNA region of the genome; and (e) located at a distance more than about 300 kb from each microRNA (miRNA) gene of the genome.

In certain embodiments, the presently disclosed methods further include measuring cleavage efficiency of a gene editing system that is delivered at the loci and selecting a locus as an GSH if the cleavage efficiency of the gene editing system at the locus is at least about 90%.

In certain embodiments, the presently disclosed methods further include measuring cleavage efficiency of a gene editing system that is delivered at the loci and selecting a locus as an GSH if the cleavage efficiency of the gene editing system at the locus is at least about 95%.

In certain embodiments, the gene editing system is a CRISPR gene editing system.

In certain embodiments, the presently disclosed methods further include measuring expression of a transgene that is integrated at the loci and selecting a locus as an GSH if the transgene integrated at the locus is expressed at a detectable level.

In certain embodiments, the transgene encodes a molecule. In certain embodiments, the molecule is an antigen-recognizing receptor that binds to an antigen. In certain embodiments, the antigen-recognizing receptor is selected from a chimeric antigen receptor (CAR), a T-cell receptor (TCR), a chimeric co-stimulating receptor (CCR), and a TCR like fusion molecule. In certain embodiments, the antigen-recognizing receptor is a chimeric antigen receptor (CAR).

In certain embodiments, the presently disclosed methods further include determining whether the loci comprise a pseudogene and selecting a locus as an GSH if the locus comprises a pseudogene.

In certain embodiments the presently disclosed methods include determining the chromatin accessibility of the loci across the genome and selecting a locus as an GSH if the locus has higher chromatin accessibility than about 90% of the plurality of loci screened.

In certain embodiments, the chromatin accessibility is determined by an Assay for Transposase-Accessible Chromatin with high-throughput sequencing (ATAC-seq).

In certain embodiments, the presently disclosed methods further include comprising selecting a locus as an GSH if the locus is located at a distance of about 5 kb from an ATAC-seq peak. In certain embodiments, the ATAC-seq peak is present in both resting and activated states of a cell.

In certain embodiments, the presently disclosed methods further include selecting a locus as an GSH if the locus is located at a distance of up to about 250 kb from at least one gene that is activated and expressed in both resting and activated states of a cell.

In certain embodiments, the presently disclosed methods further include selecting a locus as an GSH if ATAC-seq peaks are present on both sides of the locus. In certain embodiments, the ATAC-seq peaks is located at a distance of up to about 250 kb from the GSH. In certain embodiments, the ATAC-seq peaks are present in both resting and activated states of a cell.

In certain embodiments, the cell is a T cell. In certain embodiments, the cell is a T cell.

### 4. BRIEF DESCRIPTION OF THE DRAWINGS

FIG. 1 depicts the experimental scheme to obtain ATAC-seq atlas for human T cell genome.

FIG. 2 depicts the experimental scheme to identify candidate genomic safe harbors (GSHs).

FIG. 3 depicts the flowchart for identification of candidate GSHs for testing. Genomic safe harbor (GSH) atlas without pseudogene i.e. where pseudogenes were excluded from GSH and considered as genes, comprised 233 Mbp while with pseudogene atlas comprised 312 Mbp. The T-cell ATAC-seq atlas comprised of 21566 ATAC-seq peaks that were reproducible across all cell types and donors tested. GSH atlas with pseudogene and T cell ATAC-seq atlas were overlaid to identify ATAC-seq peaks that had an GSH within 5 kb. 379 such GSH peaks were identified which were then scored for their signal intensities as reads per million averaged across all cell types and donors and then ranked based on the average peak signal intensities. The top 6 highest intensity sites highlighted by inner box were tested for their cleavage efficiencies and transgene expression. The top 20 sites highlighted by outer box are tested and characterized them to identify the best GSHs for expression of a gene of interest.

FIG. 4 depicts a zoomed view of a candidate GSH peak spanning 860 bp (in black) and the 4 guide RNAs (gRNAs) indicated in flash signs tested for the GSH lying within the peak boundary and at the summit of the peak.

FIG. 5 provides cleavage efficiencies for all six selected GSHs. CRISPR/Cas9 cleavage efficiencies of four independent gRNAs represented by each independent symbol at each of the 6 top GSHs. Cleavage efficiencies were determined through analysis of the sequencing data after PCR amplification of the site and sequencing of the amplicon via deep sequencing or Sanger sequencing. Results are shown for all 24 gRNAs tested with the peripheral blood derived human T cells from one donor.

FIG. 6 depicts CAR knock-in construct at first three GSHs. CRISPR/Cas9-targeted CAR gene cassette for integration into the first three top GSHs. The top part illustrates representative GSH peak with gRNA cleavage site indicated by flash signs; the bottom part illustrates rAAV6 donor cassette containing a 1928z1xx CAR (shown on the right) driven by an Elongation factor 1 alpha (EF1α) flanked by homology arms for the GSH peak.

FIG. 7 depicts the experimental scheme for CAR integration and preparation of CAR integrated T cells for proliferation assay.

FIGS. 8A-8B provide CAR expression at GSHs overtime in culture upon multiple antigenic stimulation. FIG. 8A illustrates the experimental scheme for weekly antigenic stimulation of CAR⁺ T cells. CAR⁺ T cells were plated onto 3T3 cells expressing CD19 at day 7 after transduction and profiled for CAR expression at day 0, 4, 7 and 14 days after initial stimulation. Flow cytometry for CAR expression on day 0, 7 and 14 was performed just before plating onto 3T3 cells. FIG. 8B provides CAR expression profile (MFI) of CAR⁺ T cells with CAR integrated at GSH 1, 2 and 3 and TRAC over two weekly stimulations.

FIGS. 9A-9E provide identification and targeting of Genomic Safe Harbors (GSH). Left panel of FIG. 9A depicts the flowchart used for identification of accessible candidate GSHs and right, mean signal intensities of ATAC-seq peaks associated with the 379 GSHs (without pseudogene list) ranked by their signal intensities; RPM, reads per million. The top 6 highest intensity GSHs highlighted by the black box were tested for their cleavage efficiencies and transgene expression. Error bars are ±s.d. of n=7 cell replicates. FIG. 9B is a volcano plot depicting the 379 GSHs centered on the GSH peak with a 5 kb region on each side of the peak in each of the 7 cell samples. Peaks are arranged in decreasing order of their highest (peak summit) signal intensities. The gray shades indicates the value of signal intensities as given in the key to the right. The GSH coverage column depicts the region that falls under GSH criteria 1-6 in light gray and the region that falls outside the criteria in dark gray. FIG. 9C is an analysis of cleavage efficiency at top 6 candidate GSHs. Top, A zoomed-in view of an example candidate GSH peak spanning 1865 bps and the 4 gRNAs tested for the GSH at the summit of the peak. Bottom, CRISPR/Cas9 cleavage efficiencies of 4 independent gRNAs (each independent symbol) at the 6 top GSHs. Cleavage efficiencies were determined through analysis of the sequencing data after PCR amplification of the site and sequencing of the amplicon via deep sequencing or Sanger sequencing. Results are shown for all 24 gRNAs tested with peripheral blood derived human T cells from one donor. See FIG. 13B for data from additional donors for one selected gRNA per GSH. FIG. 9D is analysis of cleavage efficiency within vs outside an GSH peak. 4.5 kb genomic region within and around GSH 1 peak showing gRNAs targeted and their respective cleavage efficiencies. Distances from the edge of the peak are given at the top along with the name of each gRNA; R.B.: right boundary of peak; L.B.: left boundary of peak. Cleavage efficiency values are shown as symbols for two independent T cell donors, Dotted line represents mean of the two values. Numbers on x axis indicate distance in base pairs for the entire 4.5 kb region. FIG. 9E depicts the cytotoxicity assay for the CD19-CAR targeted at GSHs 1, 2, 3 and TRAC locus using firefly luciferase (FFL)-expressing NALM-6 as targets cells. Data is shown as mean±s.d of 3 technical replicates from the same donor.

FIGS. 10A-10C provide in vitro assessment of GSH-CAR functionality. FIG. 10A depicts experimental schema for weekly antigenic stimulation of purified CAR⁺ T cells at day 7 after transduction. Flow cytometry for CAR expression on day 0, 7 and 14 was performed just before plating onto CD19-aAPCs; aAPCs: artificial Antigen presenting cells. FIG. 10B depicts CAR expression profile of CAR⁺ T cells with CAR integrated at GSH 1, 2, 3 and TRAC locus; UT: Untransduced cells used as controls. Right, Median fluorescence intensity of CAR expression for all histograms. FIG. 10C depicts proliferation in response to weekly antigenic stimulation for the CAR T cells in FIG. 10B shown as cumulative fold change in T cell numbers.

FIGS. 11A-11C provide characterization of GSHs and association with function. FIG. 11A, 1 Mbp region centered on the GSH peak for GSHs 1-6 as well as GSHs 7, 12, 20 and 30 are shown. Refseq coding genes are GAP43, LSAMP, TSPAN13, AGR2, AGR3, AHR, TMEM161B, NECTIN3, TSC22D1, NUFIP1, GPALPP1, GTF2F2, TPT1, SLC24A30, ZNF425, ZNF398, ZNF282, ZND212, ZNF777, ZNF746, ZNF467, ZNF862, ACTR3C, SIPA1L2, MAP10, NTPCR, PCNX2, ZNF338, BMS2, noncoding genes are KCCAT333, NR_110013, NR_039993, NR_105020, LINC00461, NECTIN3-AS1, TSC22D1-AS1, LINC0030, NR_120424, LINC01745, LINC00839, LINC01518, LOC283028, NR_134479, LINC01264, NR_125822, pseudogenes are BRWD1P3, RPSAP29, ZNF767P, SSPO, LOC441666, CCNYL2, and ZNF378P, RPSAP74, KRT8P4. ATAC-seq peaks in activated cells obtained from the presently disclosed data (donor 2 is used as a representative) are next to “Activated”. ATAC-seq peaks in resting cells, obtained from data in Corces et al., are next to “Resting”. The signal intensity for both sets of data were scaled to the same range for all panels. FIG. 11B is a summary of CAR expression over multiple weekly stimulations, surrounding ATAC-seq peaks, gene presence and expression at all 10 GSHs given in FIG. 11A. Column 2: expression in the immediate (Imm) or day 0, early or day 7 and late or day 14 stages of multiple stimulation; Column 3: Number of ATAC-seq peaks within 250 kb in activated (A) or resting (R) state; Column 4: Presence of ATAC-seq peaks in activated (A) or resting (R) state; Column 5: Peak signal intensity of neighboring ATACseq peaks. Peaks are characterized with a peak signal intensity of Hi:=/>1.5; Med:1-1.5; Lo:<1; Column 9: Gene expression in T cells (activated or resting); Column 10: Activated vs resting state gene expression. NS, non-significant; DE, differentially expressed; NA, not applicable. The GSHs are highlighted by shades based on their functionality with respect to expression over-time. GSHs 2-4 and 30, immediate expression only; GSHs 1, 5, 7, 12, 20, immediate and early expression; GSH 6, immediate, early and late expression. FIG. 11C depicts the presently disclosed criteria for GSH selection.

FIGS. 12A-12C provide analysis of correlation between cleavage efficiency and chromatin accessibility. FIG. 12A depicts cleavage efficiencies of Multiple target site specific (MTSS) gRNAs in K562 cells (data taken from Van Overbeek et al.) plotted vs maximum ATAC-seq peak signal intensities in K562 cells (data taken from ENCODE) within 200 bp of the gRNA target. The first panel represents all 127 MTSS gRNA targets and subsequent panels show MTSS gRNA targets grouped by the respective gRNA. The no. of target sites and the Spearman's correlation co-efficient between cleavage efficiency and signal intensity for the associated ATAC-seq peak for each group are given in the enclosed box in each panel. A target RPM=/>0.2 signifies presence of an ATACseq peak at site. FIGS. 12B-12C depict mean signal intensities in 7 cell replicates as in FIG. 9B and cleavage efficiencies at FIG. 12B, Low intensity GSH peaks with 2 gRNAs per site (each symbol of the same sign) and at 3 GSHs with 4 gRNAs/site identified in Papapetrou et al. not associated with an ATAC-seq peak and FIG. 12C, MTSS Group 3 gRNA targets. All were simultaneously analyzed in 3 independent T cell donors and 2 replicates of one of these donors (Donor 4_1 and 4_2), represented as different symbols. Target sites are ordered by their signal intensities. SH2 has a lncRNA gene located 1 kb away from the gRNA target; Sites 3b,c,d,h are located within a gene or very close to a gene (<1 kb away); 3g,j have a gene ˜5 kb away from the gRNA target while sites 3a,e and f are non-genic and do not have a gene located within 5 kb from the target.

FIGS. 13A-13C provide in vitro analysis of top 6 GSHs. FIG. 13A depicts an analysis of cleavage efficiency within vs outside an GSH peak. 4.5 kb genomic region within and around GSH 5 peak showing gRNAs targeted and their respective cleavage efficiencies. Distances from the edge of the peak are given at the top along with the name of each gRNA; R.B.: right boundary of peak; L.B.: left boundary of peak. Cleavage efficiency values are shown as symbols for two independent T cell donors, Dotted line represents mean of the two values. Numbers on x axis indicate distance in basepairs for the entire 4.5 kb region. FIG. 13B depicts CRISPR/Cas9 cleavage efficiencies with the gRNA for each of the top 6 GSHs that was used for CAR targeting with peripheral blood derived human T cells from 2 or 3 independent donors different from FIG. 9D (each independent symbol). FIG. 13C depicts flow plots of CAR expression from T cells transduced with GSH-CARs at day 3 after transduction before CAR purification indicative of integration efficiency in 3 independent experiments with n=3 different T cell donors. MFI, median fluorescence intensity. FIG. 13C depicts data from cells used in FIGS. 9G, 10B, 10C. Each panel illustrates data from all constructs performed simultaneously with an independent donor. Data is shown as cumulative fold change in T cell numbers, mean±s.d of 2 technical replicates or one sample in panels 2 and 4. h, Cytotoxicity assay performed at day 7 after CAR transduction (see schema in FIG. 9E). Each panel illustrates data from all constructs performed simultaneously with an independent donor. Data is shown as mean±s.d of 3 technical replicates.

FIGS. 14A-14F provide in vitro efficacy characterization of GSHs 7, 12, 20 and 30. FIG. 14A depicts CRISPR/Cas9 cleavage efficiencies of 2 independent gRNAs at the peak summit (each independent symbol) at the GSHs 7, 12, 20 and 30. FIG. 14B depicts Flow plots of CAR expression from T cells transduced with GSH-CARs at day 3 after transduction before CAR purification indicative of integration efficiency in one representative T cell donor. FIG. 14C depicts CAR expression profile of GSH-CARs over three weeks in 2 independent T cell donors shown in the 2 adjoining panels for GSHs 7, 12, 20 and 30, day 0 is 7 days after T cell purification i.e. day 10 as per schema in FIG. 21F. FIG. 14D and FIG. 14E depict two vertical panels show cytotoxicity assay data for all CARs shown in both panels in FIG. 14C depicts at day 0 (d) and at day 21 (e). Data is shown as mean±s.d of 3 technical replicates. FIG. 14F depicts two vertical panels show proliferation in response to weekly antigenic stimulation for the cells in both panels in FIG. 14C.

### 5. DETAILED DESCRIPTION

The present disclosure provides methods for identifying GSHs in a genome (e.g., a human genome), e.g., for targeted integration. The methods include screening a plurality of loci within a genome, evaluating the position of the loci, and identifying a locus as an GSH if such locus meets the following criteria: (a) located at a distance of more than about 50 kb from the 5′ end of each gene of the genome; (b) located at a distance of more than about 300 kb from each cancer-related gene of the genome; (c) located outside each gene transcription unit of the genome; locate outside of each ultra-conserved region of the genome; (d) located outside of each non-coding RNA region of the genome; and (e) located at a distance more than about 300 kb from each microRNA (miRNA) gene of the genome. It is based, at least in part, on the discovery that transgenes integrated into the GSHs identified by the methods disclosed herein have reliable and stable expressions.

Non-limiting embodiments of the present disclosure are described by the present specification and Examples.

For purposes of clarity of disclosure and not by way of limitation, the detailed description is divided into the following subsections:
5.1 Definitions; and
5.2 Methods for identifying GSHs in genomes.

### 5.1 Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, a “genomic safe harbor” or “GSH” refers to a chromosome location where an integrated transgene can be predictably expressed without adversely affecting endogenous gene structure or expression. In certain embodiments, integrating a transgene at the GSH does not alter cell behavior and/or promote malignant transformation of the host cell or the organism. In certain embodiments, the GSH permits sufficient transgene expression to yield desirable levels of protein or non-coding RNA encoded by the transgene.

As used herein, a “transgene” refers to an exogenous DNA sequence that is introduced into the genome of a cell, including a genetically modified cell. In certain embodiment, the transgene encodes a non-coding RNA. In certain embodiment, the transgene encodes a polypeptide. In certain embodiments, the polypeptide is a therapeutic polypeptide. In certain embodiments, the polypeptide is not expressed in the genetically modified cell. In certain embodiments, the polypeptide is endogenously expressed in the genetically modified cell in an amount that does not have an intended biological or therapeutic effect.

As used herein, the term “locus” refers to the specific physical location of a DNA sequence (e.g., a genomic safe harbor, a gene, a pseudogene, an extragenic region) on a chromosome.

### 5.2 Methods for Identifying GSHs in a Genome

The present disclosure provides methods for identifying GSHs in a genome, including a genome of a human or a non-human organism. In certain embodiments, the methods comprise identifying GSHs in a genome based on the positions of the loci within the genome, DNA accessibility of the loci, and/or chromatin accessibility of the loci.

Non-limiting examples of non-human organisms that can be used with the presently disclosed subject matter include animals, plants, fungi, and yeasts. Non-limiting examples of animals that can be used with the presently disclosed subject matter include mammals, birds, reptiles, fish, and insects. Non-limiting examples of mammals that can be used with the presently disclosed subject matter include mice, rats, hamsters, guinea pigs, rabbits, dogs, cats, sheep, pigs, goats, cattle, horses, monkeys, and apes.

### 5.2.1. Positional Criteria

In certain embodiments, the methods disclosed herein for identifying an GSH comprise: screening a plurality of loci within a genome, evaluating the position of the loci, and identifying a locus as an GSH if the locus meets the positional criteria disclosed herein.

In certain embodiments, the positional criteria include selecting a locus that is located in an extragenic region, thus avoiding disrupting at least one endogenous gene. In certain embodiments, a locus that is located in an extragenic region includes a locus that is not located in close proximity from the 5′ end of each gene of the genome. In certain embodiments, a locus that is not located in close proximity from the 5′ end of each gene of the genome includes a locus that is located at a distance of at least about 50 kb, at least about 60 kb, at least about 70 kb, at least about 80 kb, at least about 90 kb, or at least about 100 kb from the 5′ end of each gene of the genome. In certain embodiments, a locus that is not located in close proximity from the 5′ end of each gene of the genome includes a locus that is located at a distance of more than about 50 kb from the 5′ end of each gene of the genome.

In certain embodiments, selecting a locus that is located in an extragenic region further includes selecting a locus that is located outside of each non-coding RNA region of the genome. In certain embodiments, a non-coding RNA (ncRNA) is a functional RNA molecule that is transcribed from DNA but not translated into proteins. Non-limiting examples of ncRNAs include microRNAs (miRNAs), small interference RNAs (siRNAs), PIWI-interacting RNAs (piRNAs), long non-coding RNAs (lncRNAs), Mt_rRNA, Mt_tRNA, misc.RNA, rRNA, scRNA, snRNA, snoRNA, ribozyme, sRNA, and scaRNA.

In certain embodiments, selecting a locus that is located in an extragenic region further includes selecting a locus that is not located in close proximity from each miRNA gene of the genome. In certain embodiments, a locus that is not located in close proximity from each miRNA includes a locus that is located at a distance of at least about 300 kb, at least about 320 kb, more than about 350 kb, more than about 380 kb, or more than about 400 kb from each miRNA gene of the genome.

In certain embodiments, a locus that is not located in close proximity from each miRNA includes a locus that is located at a distance of more than about 300 kb from each miRNA gene of the genome.

A major risk posed by transgene integration is that of malignant transformation, in which transgene integration may activate expression of an oncogene, and thus may cause or facilitate cancer. In certain embodiments, the positional criteria further include selecting a locus that is not located in proximity to at least one cancer-related gene. In certain embodiments, a locus that is not located in proximity to a cancer-related gene includes a locus that is located at least about 300 kb, at least about 350 kb, at least about 400 kb, at least about 450 kb, at least about 500 kb, at least about 550 kb, at least about 600 kb, at least about 650 kb, or at least about 700 kb from each cancer-related gene of the genome.

In certain embodiments, cancer-related genes include oncogenes or any genes that are known to play a role in cancer initiation, growth, metastasis, or any aspects of cancer in humans or non-humans.

In certain embodiments, the positional criteria further include selecting a locus that is located outside transcription units, to avoid disruption of the expression of at least one endogenous coding gene. In certain embodiments, the methods disclosed herein comprise selecting a locus that is located outside each gene transcription unit of the genome. A transcription unit refers to a segment of DNA that is transcribed into an RNA molecule. In certain embodiments, the transcription unit includes at least one gene. In certain embodiments, the transcription unit includes at least two genes.

In certain embodiments, the positional criteria include selecting a locus that is located outside of each ultra-conserved region of the genome. An ultra-conserved element or an ultra-conserved region is a segment of DNA that is over about 100 bps in length, and is over about 95% conserved in human, rat, mouse, chicken and dog genomes and significantly conserved in the fish genome. In certain embodiments, the ultra-conserved element or the ultra-conserved region is a class of genetic elements that are more highly conserved among human, rat, mouse, chicken, dog, and fish than proteins. In certain embodiments, these genetic elements may be essential for the ontogeny of mammals and other vertebrates. Altering the copy number of ultra-conserved elements can be deleterious and can be associated with cancer. Thus, selecting a locus that is located outside of each ultra-conserved region of the genome can avoid disruption of ultra-conserved regions and any adverse effects associated with the disruption.

In certain embodiments, the methods disclosed herein for identifying a genomic safe harbor (GSH), comprise: (i) screening a plurality of loci within a genome, (ii) evaluating the position of the loci, and (iii) identifying a locus as an GSH if the locus is: (a) located at a distance of more than about 50 kb from the 5′ end of each gene of the genome; (b) located at a distance of more than about 300 kb from each cancer-related gene of the genome; (c) located outside each gene transcription unit of the genome; (d) locate outside of each ultra-conserved region of the genome; (e) located outside of each non-coding RNA region of the genome; and (f) located at a distance more than about 300 kb from each microRNA (miRNA) of the genome.

In certain embodiments, the methods disclosed herein further comprise determining whether the loci comprise a pseudogene, and selecting a locus as an GSH if the locus comprises a pseudogene. In certain embodiments, pseudogenes are segments of DNA that have homology to protein coding genes but generally suffer from a disrupted coding sequence. An active homologous gene of a pseudogene can be found at another locus. In certain embodiments, the pseudogenes have an intact coding sequence or an open but truncated ORF, in which case other evidence is used (for example genomic polyA stretches at the 3′ end) to classify them as a pseudogene. In certain embodiments, pseudogenes are similar or substantially similar to a functional gene but are non-functional. In certain embodiments, a pseudogene is an allele of a functional gene that has become non-functional due to the accumulation of mutations. For example, the protein coding region of the pseudogene may contain a premature stop codon, or a frameshift mutation, or an internal deletion or insertion relative to the functional gene. Because pseudogenes are non-functional but can support gene expression, selecting a pseudogene region that conforms to the presently disclosed GSH criteria allows the expression of transgenes of interest at therapeutic levels but without adversely impacting the functionality of cells.

In certain embodiments, bioinformatic techniques are used for screening a plurality of loci within a genome, evaluating the position of the loci, and identifying a locus as an GSH that meets the positional criteria disclosed herein. Non-limiting examples of bioinformatic techniques that can be used with the presently disclosed subject matter include trimmomatic, MACS2, and Bowtie2.

### 5.2.2 DNA and Chromatin Accessibility Criteria

In certain embodiments, the methods disclosed herein for identifying an GSH further include evaluating the DNA accessibility of the loci, and selecting a locus that has high DNA accessibility such that the locus has higher chromatin accessibility than about 90% of the loci screened. High DNA accessibility is associated with reliable and stable expression of a transgene, which may be important for the downstream application of a genetically modified cell.

In certain embodiments, evaluating DNA accessibility includes measuring cleavage efficiency of a gene editing system at the loci. In certain embodiments, evaluating DNA accessibility further includes selecting a locus as an GSH if the cleavage efficiency of the gene editing system at the locus is at least about 90%. In certain embodiments, evaluating DNA accessibility further includes selecting a locus as an GSH if the cleavage efficiency of the gene editing system at the locus is at least about 95%.

Any gene editing system known in the art for targeted integration of a transgene to a predetermined chromosomal location can be used with the methods disclosed herein. Non-limiting examples of gene editing systems that can be used with the presently disclosed methods include CRISPR/Cas systems, zinc-finger nuclease (ZFN) systems, and transcription activator-like effector nuclease (TALEN) systems.

A clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), and trans-activating crRNA (tracrRNA, binds to crRNA and forms an active complex with Cas9). The terms “guide RNA” and “gRNA” refer to any nucleic acid that promotes the specific association (or “targeting”) of an RNA-guided nuclease such as a Cas9 to a target sequence such as a genomic or episomal sequence in a cell. gRNAs can be unimolecular (comprising a single RNA molecule, and referred to alternatively as chimeric) or modular (comprising more than one, and typically two, separate RNA molecules, such as a crRNA and a tracrRNA, which are usually associated with one another, for instance by duplexing).

CRISPR/Cas9 strategies can employ a vector to transfect the host cell. The guide RNA (gRNA) can be designed for each application as this is the sequence that Cas9 uses to identify and directly bind to the target DNA in a cell. Multiple crRNAs and the tracrRNA can be packaged together to form a single-guide RNA (sgRNA). The sgRNA can be joined together with the Cas9 gene and made into a vector in order to be transfected into cells.

In certain embodiments, the gRNAs are administered to the cell in a single vector and the Cas9 molecule is administered to the cell in a second vector. In certain embodiments, the gRNAs and the Cas9 molecule are administered to the cell in a single vector. Alternatively, each of the gRNAs and Cas9 molecule can be administered by separate vectors. In certain embodiments, the CRISPR/Cas9 system can be delivered to the cell as a ribonucleoprotein complex (RNP) that comprises a Cas9 protein complexed with one or more gRNAs, e.g., delivered by electroporation (see, e.g., DeWitt et al., Methods 121-122:9-15 (2017) for additional methods of delivering RNPs to a cell).

In certain embodiments, the gene editing system is a ZFN system for integrating the transgene to the loci. The ZFN can act as restriction enzyme, which is generated by combining a zinc finger DNA-binding domain with a DNA-cleavage domain. A zinc finger domain can be engineered to target specific DNA sequences which allows the zinc-finger nuclease to target desired sequences within genomes. The DNA-binding domains of individual ZFNs typically contain a plurality of individual zinc finger repeats and can each recognize a plurality of base pairs. The most common method to generate a new zinc-finger domain is to combine smaller zinc-finger “modules” of known specificity. The most common cleavage domain in ZFNs is the non-specific cleavage domain from the type IIs restriction endonuclease FokI. ZFN modulates the expression of proteins by producing double-strand breaks (DSBs) in the target DNA sequence, which will, in the absence of a homologous template, be repaired by non-homologous end-joining (NHEJ). Such repair can result in deletion or insertion of base-pairs, producing frame-shift and preventing the production of the harmful protein (Durai et al., Nucleic Acids Res.; 33 (18): 5978-90 (2005)). Multiple pairs of ZFNs can also be used to completely remove entire large segments of genomic sequence (Lee et al., Genome Res.; 20 (1): 81-9 (2010)).

In certain embodiments, the gene editing system is a TALEN system for integrating the transgene to the loci. TALENs are restriction enzymes that can be engineered to cut specific sequences of DNA. TALEN systems operate on a similar principle as ZFNs. TALENs are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome (Boch et al., Nature Biotechnology; 29(2):135-6 (2011)).

The gene editing system disclosed herein can be delivered into the host cell using a viral vector, e.g., retroviral vectors such as gamma-retroviral vectors, and lentiviral vectors. Any suitable serotype of viral vectors can be used with the presently disclosed subject matter. Combinations of viral vector and an appropriate packaging line are suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD 114 or GALV envelope and any other known in the art. Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to deliver the gene editing system to the host cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In certain embodiments, the viral vectors are oncolytic viral vectors that target cancer cell and deliver the gene editing system to the cancer cells. Non-limiting examples of oncolytic viral vectors are disclosed in Lundstrom et al., Biologics. 2018; 12: 43-60, and the content of which is incorporated by reference herein in its entirety. In certain embodiments, the oncolytic viral vectors are selected from adenoviruses, HSV, alphaviruses, rhabdoviruses, Newcastle disease virus (NDV), vaccinia viruses (VVs), and combinations thereof.

Non-viral approaches can also be employed for delivering the gene editing system to the host cell. For example, a nucleic acid molecule can be introduced into the host cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation and protoplast fusion. Liposomes can also be potentially beneficial for delivery of nucleic acid molecules into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically.

In certain embodiments, non-viral approaches include nanotechnology-based approaches, which use non-viral vectors. The non-viral vectors can be made of a variety of materials, including inorganic nanoparticles, carbon nanotubes, liposomes, protein and peptide-based nanoparticles, as well as nanoscale polymeric materials. Riley et al., Nanomaterials (Basel). 2017 May; 7(5): 94 reviews nanotechnology-based methods for delivery of a nucleic acid molecule to a subject, the content of which is incorporated as reference in its entirety.

Transgene to be delivered into the cell using the gene editing system can be ssDNA or dsDNA, depending on the delivery methods.

In certain embodiments, evaluating DNA accessibility includes measuring the expression of a transgene that is integrated at the locus. In certain embodiments, evaluating DNA accessibility further includes selecting a locus as an GSH if the transgene expression at the locus is detectable. In certain embodiments, measuring the expression of a transgene includes genetically modifying a cell to integrate a transgene at a locus, culturing the cell under conditions that favor the expression of the transgene, and measuring the transgene expression of the cell.

In certain embodiments, the transgene encodes a protein, or a non-coding RNA. In certain embodiments, the transgene expression includes transgene RNA expression or transgene protein expression. Any suitable techniques known in the art for measuring RNA and protein levels can be used with the presently disclosed methods. In certain embodiments, techniques for measuring mRNA levels include, but not limited to, real-time PCR (RT-PCR), quantitative PCR, quantitative real-time polymerase chain reaction (qRT-PCR), fluorescent PCR, RT-MSP (RT methylation specific polymerase chain reaction), PicoGreen™ (Molecular Probes, Eugene, Oreg.) detection of DNA, radioimmunoassay or direct radio-labeling of DNA, in situ hybridization visualization, fluorescent in situ hybridization (FISH), microarray.

In certain embodiments, techniques for measuring protein levels include, but are not limited to, flowcytometry, mass spectrometry techniques, 1-D or 2-D gel-based analysis systems, chromatography, enzyme linked immunosorbent assays (ELISAs), radioimmunoassays (RIA), enzyme immunoassays (EIA), Western Blotting, immunoprecipitation and immunohistochemistry.

In certain embodiments, evaluating DNA accessibility further includes selecting a locus as an GSH if the transgene expression is sustainable, for example, the transgene expression is detectable consistently or stably for a period of time. In certain embodiments, the methods disclosed herein include selecting a locus as an GSH if the transgene expression is detectable for at least about 1 week, at least about 2 weeks, at least about 3 weeks, at least about 4 weeks, at least about 5 weeks, at least about 6 weeks, at least about 7 weeks, or at least about 8 weeks after its integration to the cell. In certain embodiments, the expression of the transgene is inducible, in which the expression of the transgene is only initiated upon contacting the cell with a stimuli that induces the expression of the transgene. In certain embodiments, the methods disclosed herein include selecting a locus as an GSH if the inducible transgene expression is detectable for at least about 1 week, at least about 2 weeks, at least about 3 weeks, at least about 4 weeks, at least about 5 weeks, at least about 6 weeks, at least about 7 weeks, or at least about 8 weeks after contacting the cell with the stimuli that induces the expression of the transgene.

In certain embodiments, the transgene encodes an antigen-recognizing receptor that binds to an antigen. In certain embodiments, the antigen-recognizing receptor is selected from the group consisting of a chimeric antigen receptor (CAR), a T-cell receptor (TCR), a chimeric co-stimulating receptor (CCR), and a TCR like fusion molecule. In certain embodiments, the antigen-recognizing receptor is a chimeric antigen receptor (CAR). In certain embodiments, the method comprises measuring the expression of the CAR about at least about 12 hours from the antigen stimulation. In certain embodiments, the method comprises measuring the expression of the CAR about no later than about 5 weeks, about 4 weeks or about 130 days from the antigen stimulation. In certain embodiments, the CAR expression is measured about four (4) days from the antigen stimulation. In certain embodiments, the CAR expression is measured about one week from the antigen stimulation. In certain embodiments, the CAR expression is measured about two weeks from the antigen stimulation.

In certain embodiments, the methods disclosed herein for identifying an GSH further include evaluating the chromatin accessibility of the loci, and selecting a locus that has high chromatin accessibility. In certain embodiments, chromatin accessibility of a locus is important for the cleavage efficiency of editing system as well as expression of the transgene integrated at the locus. Low chromatin accessibility of a locus can result in lower efficiency of editing at the locus and low expression of the transgene integrated at the locus.

Non-limiting methods for evaluating chromatin accessibility include micrococcal nuclease (MNase)-assisted isolation of nucleosomes sequencing (MNase-seq), DNase I hypersensitive sites sequencing (DNase-seq), formaldehyde-assisted isolation of regulatory elements sequencing (FAIRE-seq), and assay for transposase-accessible chromatin using sequencing (ATAC-seq). Tsompana et al., Epigenetics Chromatin (2014); 7:33 reviews tools for evaluating chromatin accessibility, content of which is incorporated herein by reference.

In certain embodiments, the chromatin accessibility of the loci is evaluated by ATAC-seq. In certain embodiments, the methods disclosed herein include selecting a locus as an GSH if the locus is located at a distance of up to about 10 kb, up to about 9 kb, up to about 8 kb, up to about 7 kb, up to about 6 kb, up to about 5 kb, up to about 4 kb, up to about 3 kb, up to about 2 kb, or up to about 1 kb from an ATAC-seq peak or within an ATAC-seq peak. In certain embodiments, the methods disclosed herein include selecting a locus as an GSH if the locus is located within an ATAC-seq peak. In certain embodiments, the ATAC-seq peak is present in both resting and activated states of cells (e.g., T cells).

In certain embodiments, the chromatin accessibility of the loci is evaluated by the presence of and expression of surrounding genes in resting and activated state of a cell (e.g., a T cell). In certain embodiments, the methods disclosed herein include selecting a locus as an GSH if the locus is located at a distance of up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb, from at least one gene that is activated and expressed in resting and/or activated states of cells (e.g., T cells). In certain embodiments, the methods disclosed herein include selecting a locus as an GSH if the locus is located at a distance of up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb, from at least one gene that is activated and expressed in both resting and activated states of cells (e.g., T cells). In certain embodiments, the locus is located at a distance of up to about 250 kb from at least one gene that is activated and expressed in both resting and activated states of cells (e.g., T cells).

In certain embodiments, the chromatin accessibility of the loci is evaluated by the presence of ATAC-seq peaks surrounding the targeted site on one or both sides. In certain embodiments, the chromatin accessibility of the loci is evaluated by the presence of ATAC-seq peaks surrounding the targeted site on both sides. In certain embodiments, the methods disclosed herein include selecting a locus as an GSH if the locus is located up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb from ATAC-seq peaks that are present in the activated and/or resting states of cells (e.g., T cells). In certain embodiments, the methods disclosed herein include selecting a locus as an GSH if the locus is located up to about 500 kb, up to about 450 kb, up to about 400 kb, up to about 350 kb, up to about 300 kb, up to about 250 kb, up to about 200 kb, up to about 150 kb, up to about 100 kb, or up to about 50 kb from ATAC-seq peaks that are present in both the activated and resting states of cells (e.g., T cells). In certain embodiments, the locus is located up to about 250 kb from ATAC-seq peaks that are present in both the activated and resting states of cells (e.g., T cells).

### 6. EXAMPLE

The presently disclosed subject matter will be better understood by reference to the following Example, which is provided as exemplary of the presently disclosed subject matter, and not by way of limitation.

### Example 1: Selecting GSHs for Targeted Integration and Testing Selected GSHs

Genomic Safe Harbors (GSHs) are candidates for targeted integration. Extragenic genomic safe harbors provide safe and stable therapeutic transgene expression levels. Thus, there is a need to find genomic safe harbors for highly efficient and reproducible specific targeting in cells.

Candidate GSHs were determined if they met the following criteria: (a) are located at a distance of more than 50 kb from 5′ end of any gene, (b) are located at a distance of more than 300 kb from any cancer-related genes, (c) are located at a distance of more than 300 kb from any miRNA, (d) are located outside of a gene transcription unit, (e) are located outside of ultra-conserved regions (UCRs), and (f) are located outside of non-coding RNAs. Further criteria for selecting candidate GSHs included efficient cleavability and optimal transgene expression, both of which are governed by DNA accessibility. In addition, chromatin accessibility was used to select candidate GSHs, e.g., whether the locus was proximate to ATAC-seq peaks.

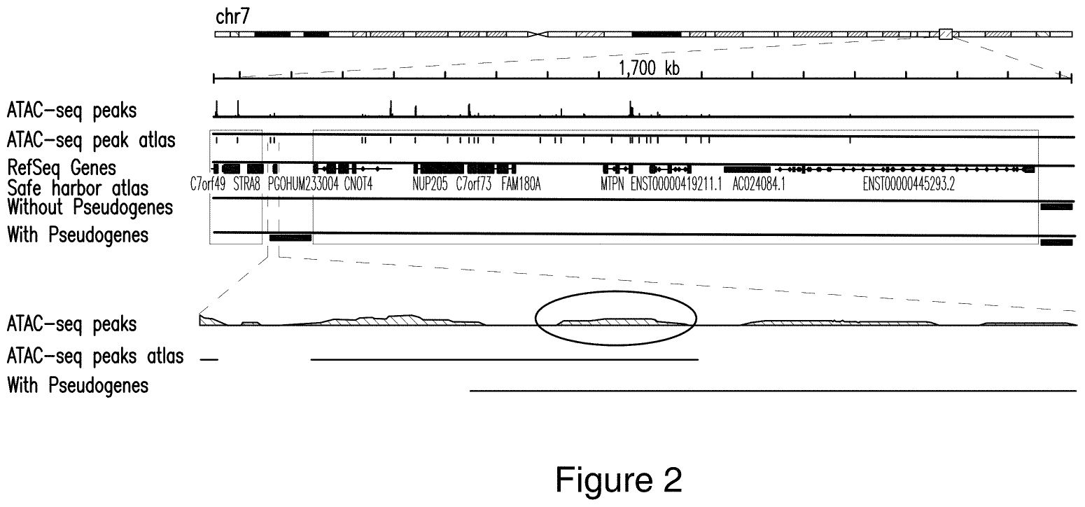

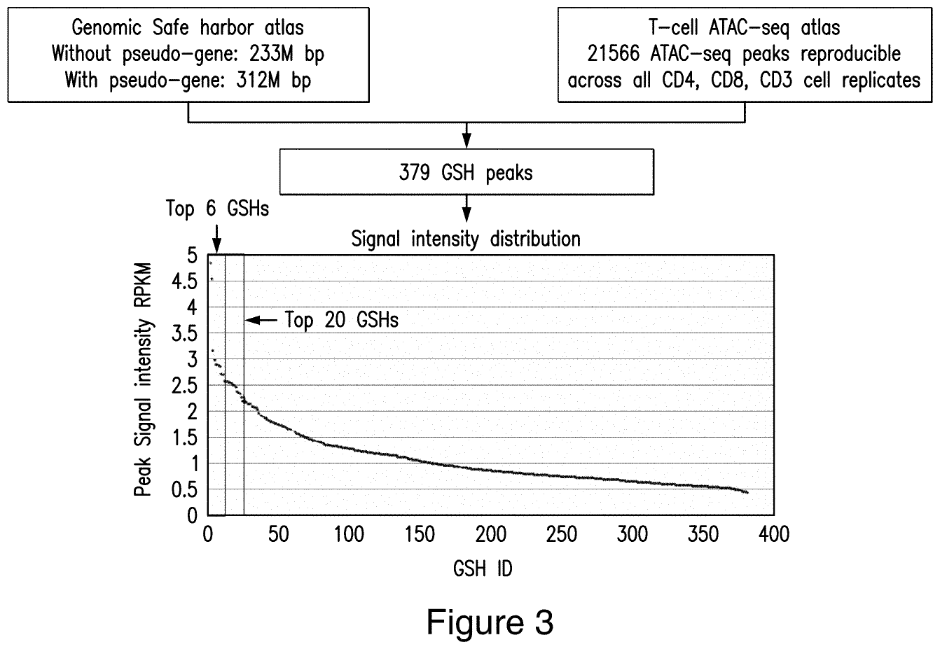

Human T cells were used to identify genomic safe harbors by employing methods disclosed herein. The ATAC-seq atlas was overlaid with GSH atlas with pseudogenes and/or GSH atlas without pseudogenes to identify GSHs (FIG. 2). The human T-cell ATAC-seq atlas comprised 21566 ATAC-seq peaks reproducible across all CD4, CD8, and CD3 cell replicates (FIG. 3). The GSH atlas without pseudogene comprised DNA regions of 233M bp in length, and the GSH atlas with pseudogene comprised DNA regions of 312M bp in length. The GSH atlas (with pseudogene) and the ATAC-seq atlas were overlaid to identify GSHs that are associated with ATAC-seq peaks. ATAC-seq peaks that had an GSH within 5 kb were identified through a custom code, and were considered as GSH peaks. GSH peaks were then scored based on peak signal intensity as replicates per million averaged across all cell types and replicates. The GSH peaks were then ranked by the average peak signal intensity scores. Loci associated with top GSH peaks were selected as top GSHs. The present example selected top 6-20 GSHs for further testing.

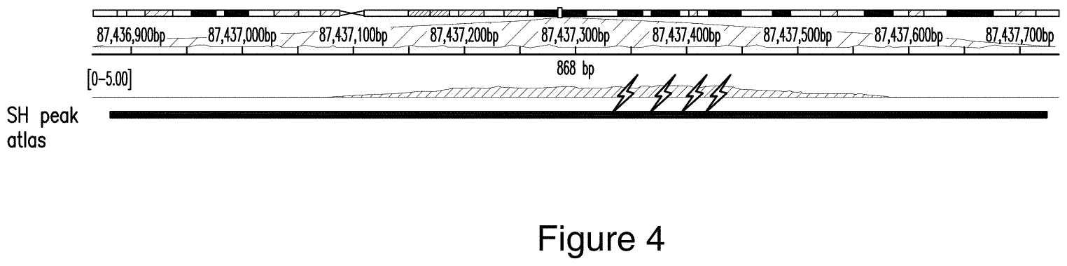

Cleavage efficiencies of the top six GSHs were analyzed by using CRISPR/Cas9 gene editing system. Cleavage efficiencies were determined through analysis of the sequencing data after PCR amplification of the site after transfecting peripheral blood derived human T cells with Cas9 mRNA and gRNAs targeting the selected six GSHs (FIG. 4).

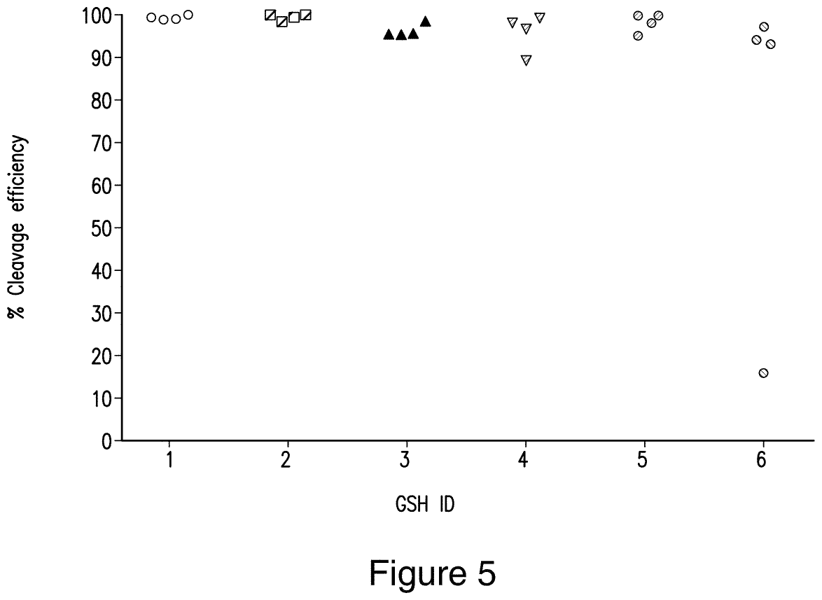

Selected top six GSHs showed high cleavage efficiencies (FIG. 5).

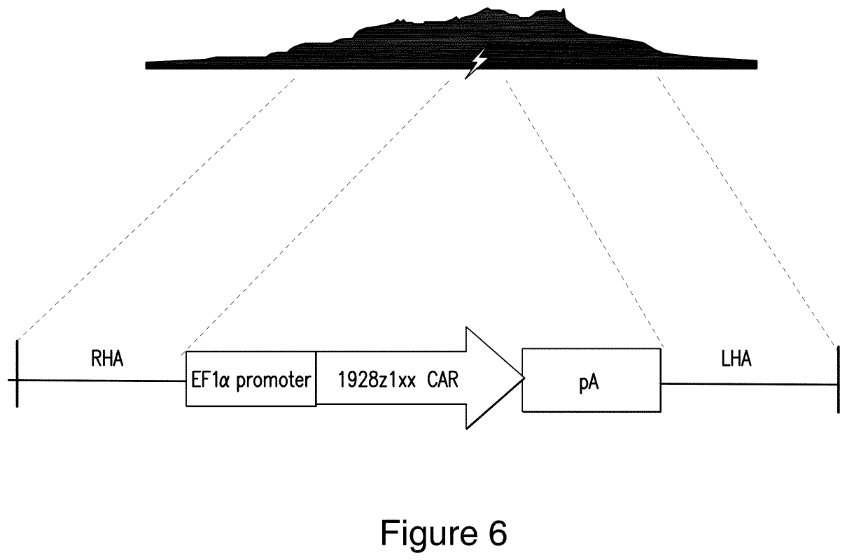

Three GSHs, GSH 1, GSH2, and GSH3, were selected as the sites for transgene integration (FIG. 6). CRISPR/Cas9-targeted CAR gene cassette was integrated into the GSHs. The cassette comprised a 1928z1xxCAR (Feucht et al., Nat. Med. 2019; 25(1):82-88) driven by an elongation factor 1 alpha (EF1α) promoter, both of which flanked by homology arms for the GSH peaks.

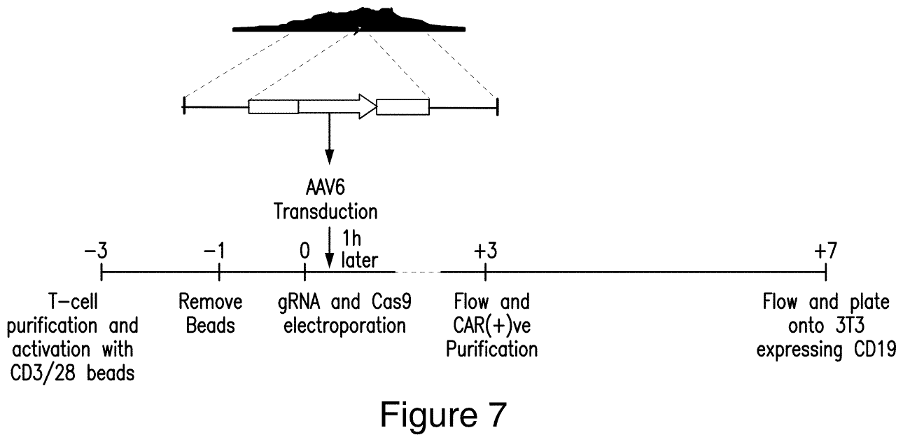

Experimental scheme was depicted in FIG. 7. Briefly, on Day −3, T cells were purified and activated with anti-CD3/CD28 beads. On Day −1, anti-CD3/CD28 beads were removed. On Day 0, gRNA and Cas9 were electroporated into the activated cells. Two hours after the electroporation, AAV6 were also transduced into cells.

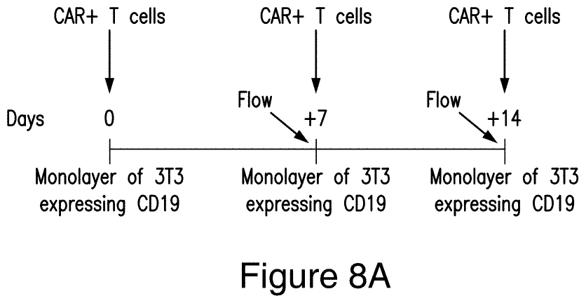

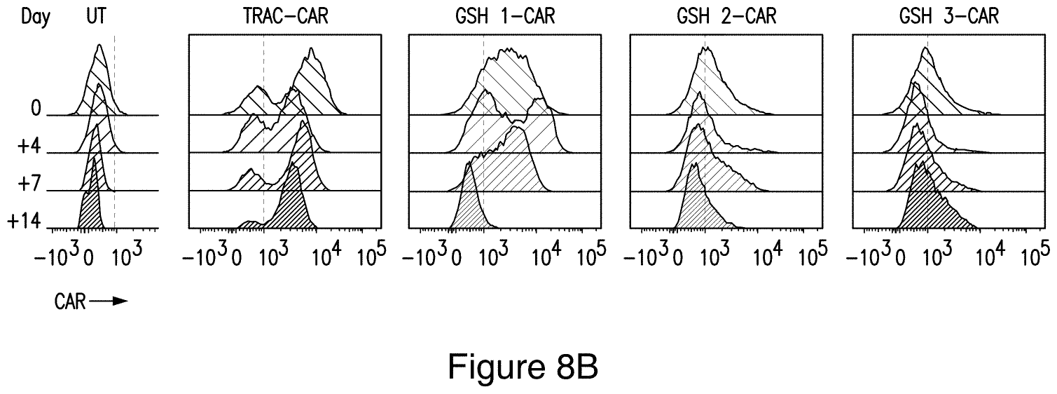

As shown in FIG. 8A weekly antigenic stimulation was applied to CAR+ T cells having CAR expressing cassette integrated at GSHs and TRAC. Untransduced cells (UT) were used as control. CAR+ T cells were plated onto 3T3 cells expressing CD19 at day 7 after transduction and profiled for CAR expression at day 0, 4, 7 and 14 days after initial stimulation. Flow cytometry for CAR expression on day 0, 7 and 14 was performed just before plating onto 3T3 cells. During the first week of stimulation, an increased CAR expression was observed on the surface of GSH-CAR T cells (FIG. 8B).

Materials and Methods

GSH Atlas Generation

All eight properties of candidate GSHs disclosed herein were applied to build a Genomic safe harbor atlas (GSH) atlas. Gene data for gene transcription units and 5′ end of any gene were obtained from GENCODE and RefSeq_NM database from NCBI. The 5′ end of a gene was calculated from the transcription start site (TSS). Data for cancer-related genes were obtained by combining oncogene lists from Bushman group allOnco list (v2) (http://www.bushmanlab.org/links/genelists), COSMIC Cancer gene census v78 (https://cancer.sanger.ac.uk/cosmic) and Cancer GeneticsWeb (http://www.cancer-genetics.org/). miRNA data was obtained from hg19 sno/miRNA track in UCSC Genome Browser and also GENCODE release 19 entries for miRNAs. UCRs in the human genome were obtained from Bejerano et al., Science 2004; 304(5675):1321-1325. And the data were downloaded from http://users.soe.ucsc.edu/˜jill/ultra.html. As the genomic coordinates used in the publication were from an older assembly, the coordinates were converted using UCSC lift genome annotations tool. Data for non-coding RNA (ncRNA) list were obtained from NONCODE v5 (www.noncode.org) and GENCODE ncRNA entries. Pseudogene annotation from GENCODE was used to either include or exclude pseudogenes from the gene list to create two atlases—Without pseudogenes and With pseudogenes. The assembly gaps as mentioned on the UCSC Genome Browser were excluded.

ATAC-Seq Atlas for Human T Cell Genome

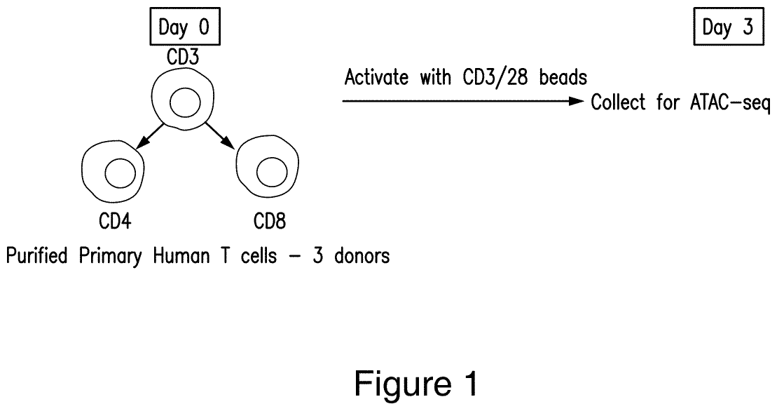

Human T cell genome was profiled for accessibility through ATAC-seq to build ATAC-seq atlas (FIG. 1). Peripheral blood mononuclear cells were obtained by density gradient centrifugation from peripheral blood of three healthy adult human volunteers. Three days after isolation and activation, the T cells were sorted into CD4 and CD8 fractions from two donors by magnetic separation through negative selection using Human CD4-biotin and Human CD8-biotin beads (Miltenyi Biotec) and anti-biotin beads (Miltenyi Biotech). CD3, CD4 and CD8 cells from two donors and only CD3 cells from third donor were collected and 50,000 cells were frozen in freezing medium (10% DMSO in FBS) for ATAC-seq analysis. ATAC-seq was performed by the Memorial Sloan Kettering Cancer Center (MSKCC) IGO core. The method used for ATAC-seq was performed as described in Buenrostro et al., Curr. Protoc. Mol. Biol. 2015; 109:21.29.1-9, with certain modifications. For example, the transposition reaction was performed at 42° C. for 45 mins for a better library preparation. All ATAC libraries were sequenced using paired-end, dual-index sequencing on a HiSeq instrument with 2×50 bp reads for at least 30 million read pairs.

Raw FASTQ reads were trimmed with trimmomatic and aligned using Bowtie2. Bam files were filtered based on map quality and PE concordance. Duplicated reads were removed and tn5 specific read shift was performed. To identify peaks, data were aggregated by each cell type, and peak summits were identified using MACS2 and filtered using a custom blacklist. IDR analysis was performed for all replicate pairs. Peaks with global IDR <0.05 were considered as reproducible peaks. 21566 ATAC-seq peaks were found to be reproducible across all cell types and replicates tested.

Guide RNA (gRNA) Design and Testing

Four gRNAs were designed and tested for each of the top 6 GSH peaks. They were designed to fall within the ATAC-seq peak and at the summit of the peak. gRNAs that had the cleavage efficiency scores (Doench scores) of more than 50, and the off-target specificity scores more than 0.2 were chosen.

2′-O-methyl 3′ phosphorothioate end modified guide RNAs (gRNAs) were synthesized by Synthego and Cas9 mRNA was synthesized by TriLink Biotechnologies. gRNAs were reconstituted at 1 μg.μl⁻¹ in sterile TE buffer.

To measure CRISPR/Cas9 mediated cleavage efficiency, CD3/CD28 beads were magnetically removed 48 hours after T cell activation was initiated. About 60-72 hours after the initial isolation and activation of T cells, T cells were electroporated with Cas9 mRNA and modified gRNA (1 μg each for 2×10⁶ cells) using the Amaxa 4D nucleofector P3 Primary Cell XKitS system (Lonza). Three days after electroporation, the cells were pelleted. gDNA was extracted from the cell pellets for PCR amplification and sequencing of respective sites for cleavage efficiency testing. Analysis of PCR amplicon sequencing data for cleavage efficiency determination was performed using CRISPresso online tool for the deep sequencing data and the ICE online tool (Synthego) for the Sanger sequencing data.

CAR Targeting

T cells were electroporated with Cas9 mRNA and gRNA in accordance with the methods described above. Recombinant AAV6 donor vectors were added to the culture one hour after electroporation at a MOI of 5×10⁵. The culture medium was changed every 2 days and was replaced with fresh medium containing 5 ng/ml interleukin-7 (IL-7) and 5 ng/ml IL-15. The cells were cultured at a concentration of 10⁶ cells per ml.

Antigen Stimulation and In Vitro Proliferation Assays

In the weekly proliferation assay, 3 days after AAV6 transduction, CAR targeted cells were purified using magnetic Biotin-SP (long spacer) AffiniPure F(ab′)2 Fragment Goat Anti-Mouse IgG, F(ab′)2 Fragment Specific antibody (Jackson ImmunoResearch), anti-biotin microbeads and MS columns (Miltenyi Biotec). The CAR+ purified cells were cultured for 4 days as described before. NIH/3T3 expressing human CD19 cells were used as artificial antigen-presenting cells (AAPCs). For weekly stimulations, 3×10⁵ irradiated CD19+ AAPCs were plated in 24-well plates 12 h before the addition of 5×10⁵ CAR+ T cells in X-vivo15 containing human serum, 5 ng ml⁻¹ interleukin-7 (IL7) and 5 ng ml⁻¹ IL15 (Peprotech). Every 2 days, cells were counted, and media was added to reach a concentration of 2×10⁶ cells per ml. For each condition, T cells were analyzed by FACS for CAR expression at time points mentioned in the respective figures. The antibody used for CAR staining was Alexa Fluor 647 AffiniPure F(ab′)2 Fragment Goat Anti-Mouse IgG, F(ab′)2 Fragment Specific (Jackson ImmunoResearch). For setting CAR MFI, Rainbow Fluorescent Particles were used (BD Biosciences).

### Example 2: Genomic Safe Harbors for CAR T Cell Engineering

The therapeutic use of genetically engineered human cells is rapidly expanding beyond gene therapy for inherited monogenic disorders to acquired disorders. Alterations of the human genome may thus not only serve to compensate for or correct mutations (Dunbar, C. E. et al., Science 359, eaan4672 (2018)) as is the case in severe combined immune deficiencies and the thalassemias, but also introduce natural or synthetic genes to reprogram cell function, as is the case for chimeric antigen receptor (CAR) therapy (June, C. H. & Sadelain, M., N. Engl. J. Med. 379, 64-73 (2018); Sadelain, M., Riviére, I. & Riddell, S., Nature 545, 423-431 (2017)). An ideal genetic treatment should provide for predictable and dependable expression of the transgene in the intended cell type, at an optimal level and stably over time, without incurring genetic adverse events. γ-Retroviral, lentiviral and transposon-based vectors are commonly used to achieve stable genetic modifications. Albeit effective ((Dunbar, C. E. et al., Science 359, eaan4672 (2018))), they all afford semi-random integration, potentially resulting in insertional mutagenesis (Craigie, R. & Bushman, F. D. Cold Spring Harb. Perspect. Med. 2, a006890 (2012); Bushman, F., Lewinski, M., Ciuffi, A., Barr, S. & Leipzig, J. Nat. Rev. Microbiol. 3, 848-858 (2005); Schwarzwaelder, K. et al. Gammaretrovirus-mediated correction of SCID-X1 is associated with skewed vector integration site distribution in vivo. 117, 2241-2249 (2007); Singh, P. K. et al. Genes Dev. 29, 2287-2297 (2015)) and variegated transgene expression (Rivella, S. & Sadelain, M. Semin. Hematol. 35, 112-125 (1998); Ellis, J. Hum. Gene Ther. 16, 1241-1246 (2005)). Furthermore, the integration of γ-retroviral and lentiviral vectors is biased towards gene loci (Craigie, R. & Bushman, F. D. Cold Spring Harb. Perspect. Med. 2, a006890 (2012); Bushman, F., Lewinski, M., Ciuffi, A., Barr, S. & Leipzig, J. Nat. Rev. Microbiol. 3, 848-858 (2005); Dunbar, C. E. Ann. N. Y. Acad. Sci. 1044, 178-182 (2005)) increasing the probability of transgene expression and also the potential to disrupt the function or expression of endogenous genes. The most dreaded consequence is oncogene activation, which may ultimately promote malignant transformation (Stein, S. et al. Nat. Med. 16, 198-204 (2010)). A prominent example of such serious adverse events are reports of leukemia occurring in patients treated with retroviral-mediated gene therapy for X-linked severe combined immunodeficiency (X-SCID) (Kohn, D. B., Sadelain, M. & Glorioso, J. C. Nat. Rev. Cancer 3, 477-488 (2003); Hacein-Bey-Abina, S. et al. J Clin Invest 118, 3132-3142 (2008); Howe, S. J. et al. J. Clin. Invest. 118, 3143-50 (2008)). Clonal expansions stopping short of leukemic transformation have occurred in both hematopoietic stem cell therapies (Cavazzana-Calvo, M. et al. Nature 467, 318-22 (2010)) and CAR T cell therapies (Shah, N. N. et al. Blood Adv. 3, 2317-2322 (2019); Fraietta, J. A. et al. Nature 558, 307-312 (2018)). The other major detrimental consequence of semi-random integration that limits the efficacy of some gene therapies is variegated and hence unpredictable transgene expression, which includes transcriptional silencing due to chromosomal position effects and heterochromatinization (Ellis, J. Hum. Gene Ther. 16, 1241-1246 (2005)).

In principle, these challenges could be overcome if the transgene were integrated at a defined genomic site that reliably provides safe and stable gene expression. Such “genomic safe harbors” (GSH) may be intra or extra-genic. Three intra- or juxta-genic sites have been proposed as potential GSH in human cells: the adeno-associated virus site 1 (AAVS1), the chemokine (CC motif) receptor 5 (CCR5) locus and the human orthologue of the mouse ROSA26 locus (Sadelain, M., Papapetrou, E. P. & Bushman, F. D. Nat. Rev. Cancer 12, 51-58 (2011); Kotin, R. M., Linden, R. M. & Berns, K. I. The EMBO journal. 11, 5071-5078 (1992); Irion, S. et al. Nat. Biotechnol. 25, 1477-1482 (2007); Lombardo, A. et al. Nat. Biotechnol. 25, 1298-1306 (2007); DeKelver, R. C. et al. Genome Res. 20, 1133-1142 (2010); Papapetrou, E. P. & Schambach, A. Mol. Ther. 24, 678-684 (2016)). These lie either within a gene thought to be dispensable or in close proximity to genes that are deemed not to pose an oncogenic threat. Their vicinity is indeed gene-rich, which may be favorable to support transgene expression but raises the risk of their trans-activation following integration of ectopic enhancer/promoter elements.

Alternatively, one may search for remote extragenic GSH (Sadelain, M., Papapetrou, E. P. & Bushman, F. D. Nat. Rev. Cancer 12, 51-58 (2011)). The presently disclosed criteria are for the retrospective identification of safe viral vector integrations at candidate GSH. The advent of site-specific nucleases now makes it possible to direct transgene integration to GSH, provided that the latter are accessible. Focusing on T cell engineering to advance cancer immunotherapy (Sadelain, M., Rivière, I. & Riddell, S. Nature 545, 423-431 (2017)), the presently disclosed subject matter showed the use of CRISPR/Cas9 to target candidate GSH, efficiently undergo homologous recombination using AAV6 vectors (Eyquem, J. et al. Targeting a CAR to the TRAC locus with CRISPR/Cas9 enhances tumor rejection. Nature 543, 113-117 (2017); Schumann, K. et al. PNAS 112, 10437-10442 (2015); Roth, T. L. et al. Nature 559, 405-409 (2018); Sather, B. D. et al. Sci. Transl. Med. 7, 307ra156 (2015)) and support sustained transgene expression. Using a CAR specific for CD19, it was demonstrated herein that one such site, termed GSH6, directed CAR expression that was as effective as the TRAC locus, an optimal locus for CAR T cell engineering (Eyquem, J. et al. Targeting a CAR to the TRAC locus with CRISPR/Cas9 enhances tumour rejection. Nature 543, 113-117 (2017)). The identification of accessible GSH in primary T cells can facilitate the generation of T cells that predictably and homogeneously express their therapeutic gene cargo, thereby enhancing the safety and efficacy of cancer immunotherapy (June, C. H. & Sadelain, M. N. Engl. J. Med. 379, 64-73 (2018)).

### Results

Identification and Targeting of GSHs

A set of 5 safety criteria previously proposed to define extragenic genomic safe harbors (GSH) based on the avoidance of chromosomal integrations posing a risk of insertional oncogenesis (Papapetrou, E. P. et al. Nat. Biotechnol. 29, 73-78 (2011)). Based on recent findings on the role of non-coding RNAs (ncRNAs) in regulating cell function (Beermann, J., Piccoli, M. T., Viereck, J. & Thum, T. Physiol. Rev. 96, 1297-1325 (2016); Esteller, M. Nat. Rev. Genet. 12, 861-874 (2011)), a sixth criterion was added to exclude disruption of known ncRNA (Table 1). Two additional criteria were added to achieve efficient site-specific transgene integration at the selected sites, requiring dependable cleavage by nucleases like Cas9 and subsequent homologous recombination, and the further need to achieve dependable and sustained transgene function (Table 1).

TABLE 1
Criteria for identification of GSH
Criteria for GSH

1. Distance of >50 kb from 5′ end of any gene
2. Distance of >300 kb from any cancer-related gene
3. Distance of >300 kb from any miRNA
4. Outside a gene transcription unit
5. Outside of Ultra-conserved regions
6. Outside of Non coding RNAs
7. Efficient cleavability
8. Reliable transgene expression and regulation

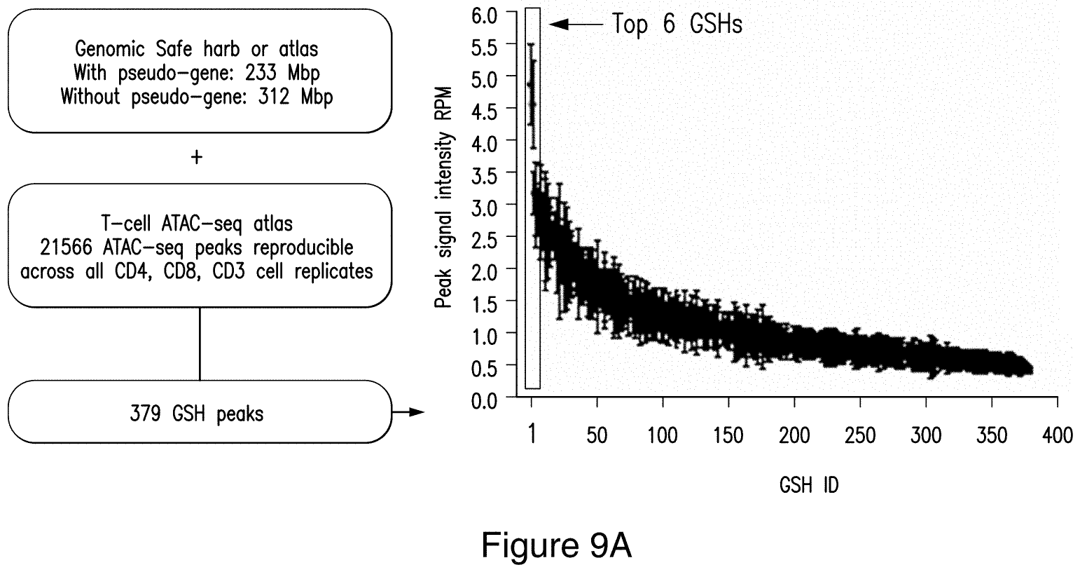

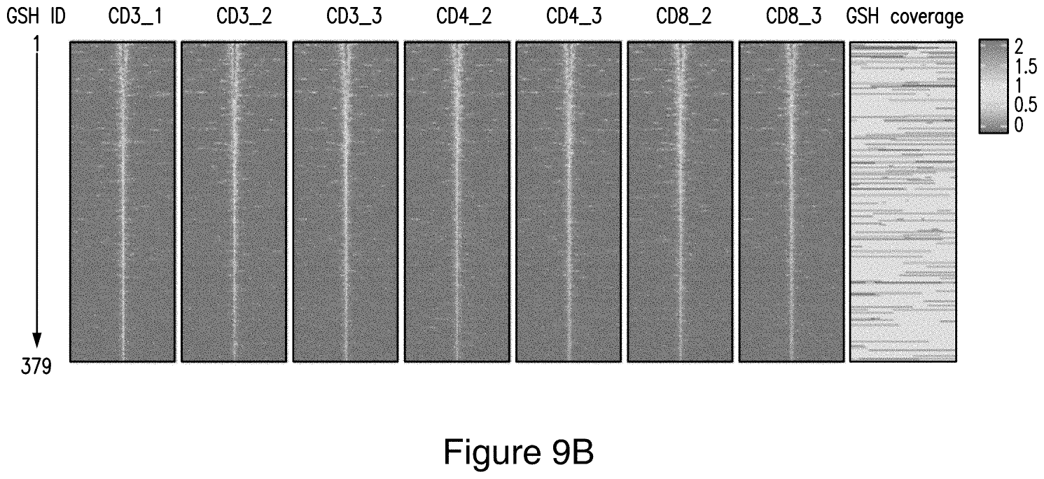

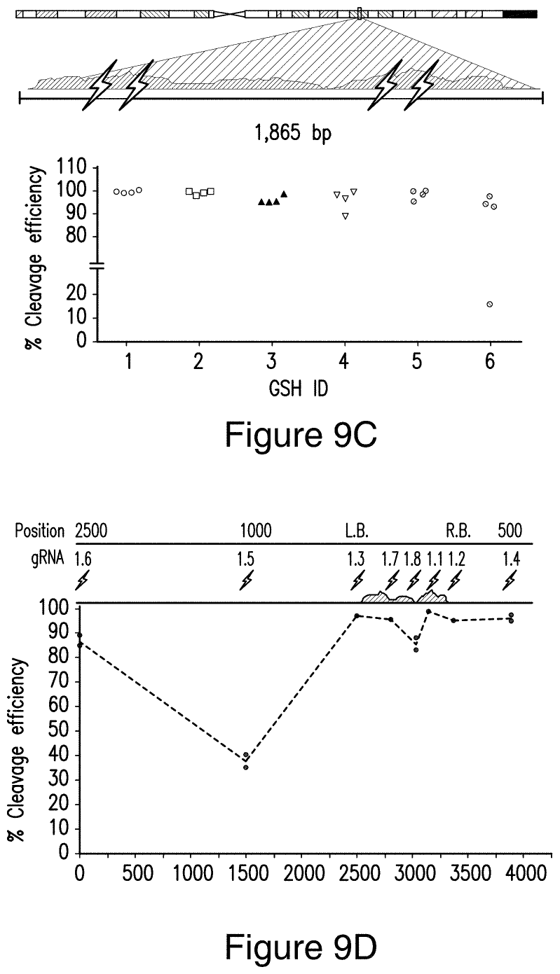

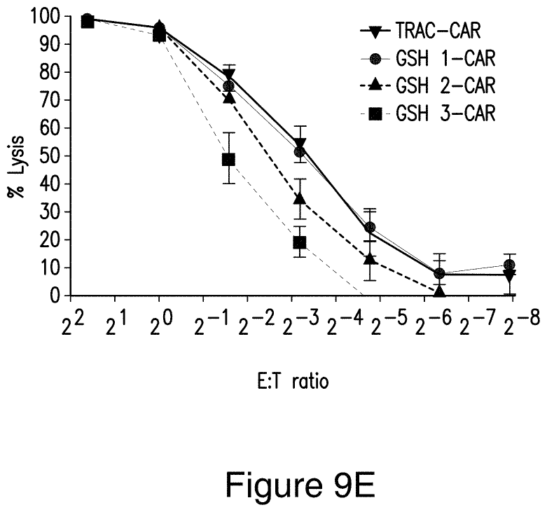

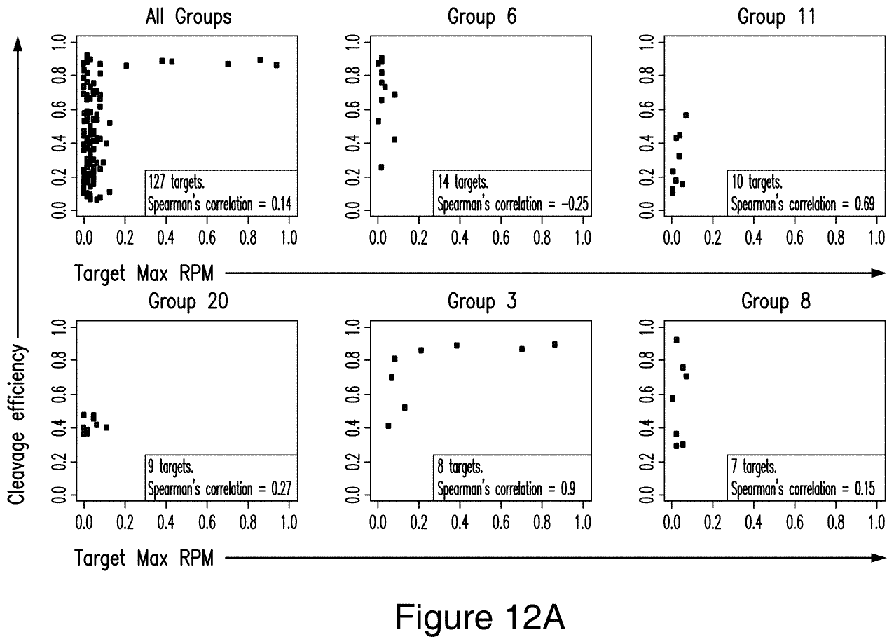

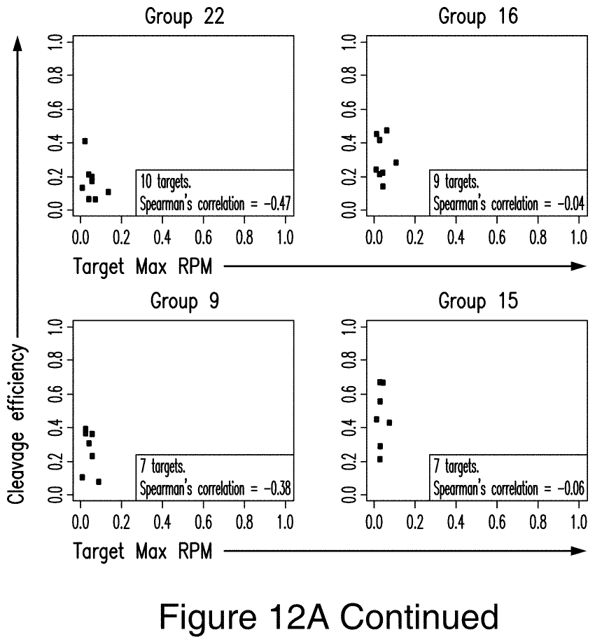

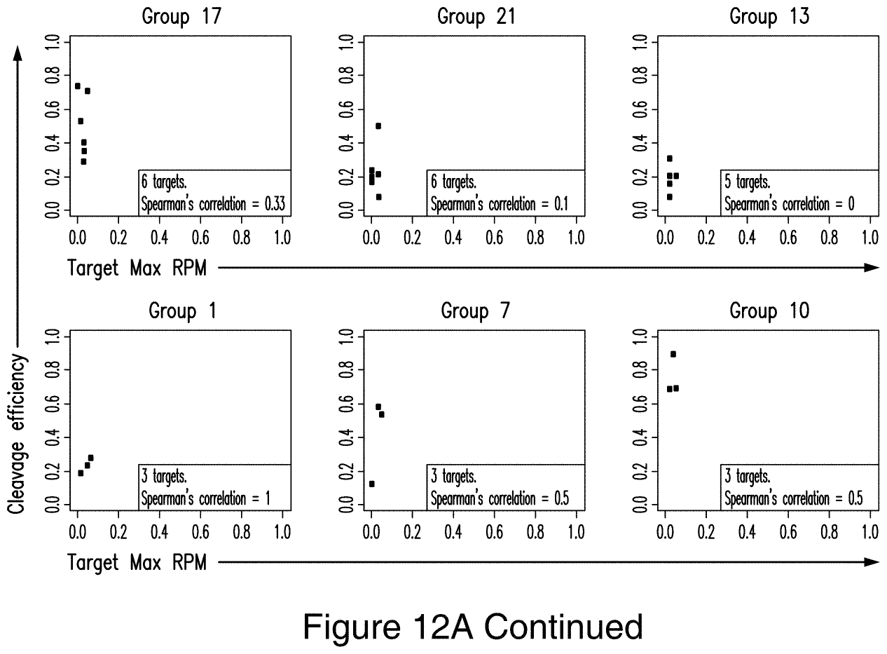

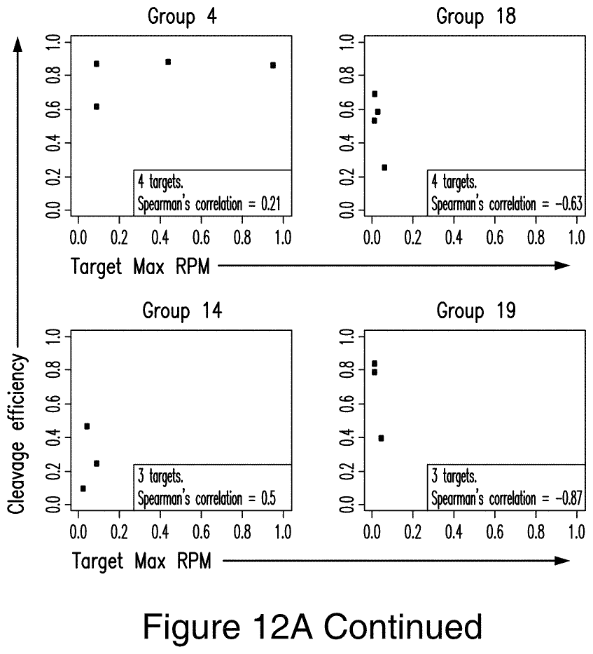

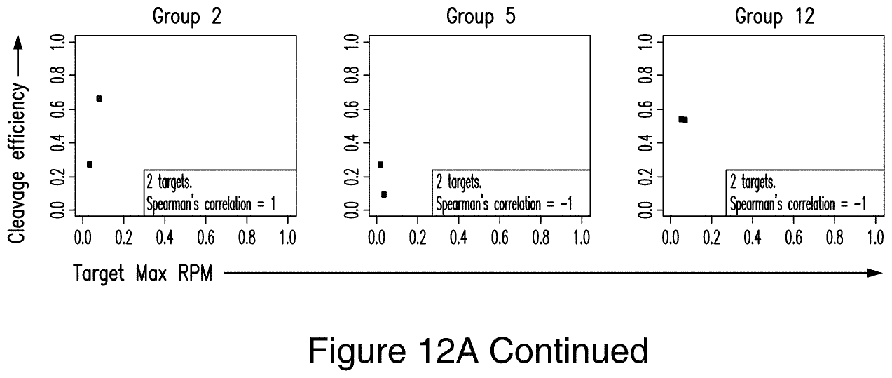

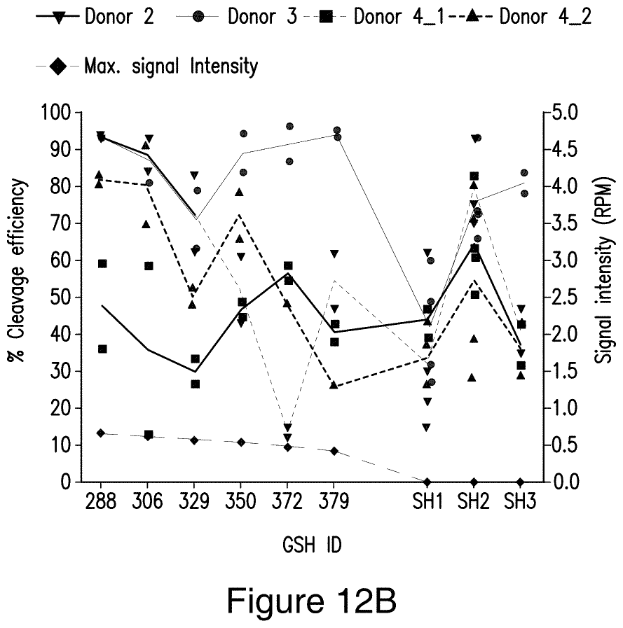

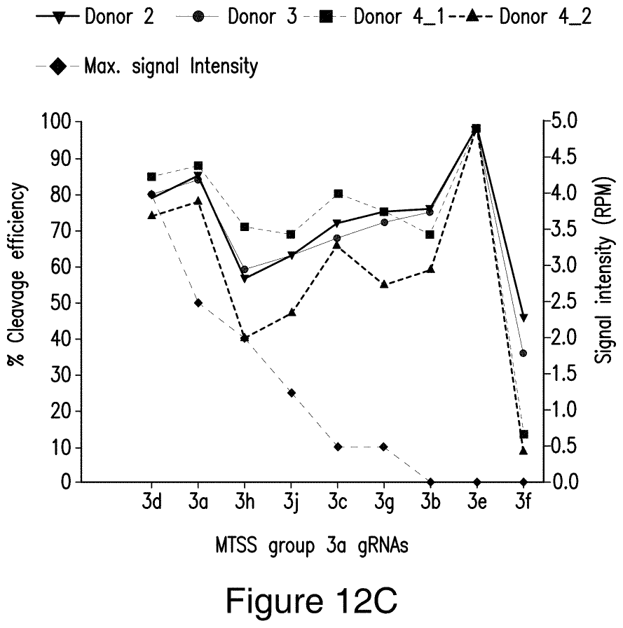

To date, the cleavage efficiencies predicted by softwares that use features of the gRNA sequence alone have been inaccurate in estimating cleavage efficiencies in a living cell (Verkuijl, S. A. & Rots, M. G. Curr. Opin. Biotechnol. 55, 68-73 (2019)). Given the very specific and dynamic chromatin environment of chromosomal DNA in living cells, the chromatin context of a genomic locus governs DNA accessibility and hence cleavability and subsequent transgene expression from that site. Analysis of data from Van Overbeek et al. (Van Overbeek, M. et al. Mol. Cell 63, 633-646 (2016)) on the activity of Cas9 suggested that a site possessing accessible chromatin indeed had a higher probability of displaying efficient cleavage (FIG. 12A). Candidate GSHs that conform to the safety and accessibility GSH criteria integrating measurable chromatin accessibility were identified. The technique of ATAC-seq (Assay for transposase accessible chromatin) was utilized to assess the genome wide chromatin accessibility of primary human T cells (Buenrostro, J. D., Wu, B., Chang, H. Y. & Greenleaf, W. J. Curr. Protoc. Mol. Biol. 109, 21.29.1-21.29.9 (2015)). ATAC-seq was performed three days after isolation and activation of primary human T cells obtained from healthy donors, a time point at which GSHs would eventually be targeted for transgene delivery. An ATAC-seq atlas was generated with the reproducible ATAC-seq peaks shared across all cell types and replicates (details in Methods) along with an GSH atlas constructed by computing regions that satisfy the first six GSH criteria. Pseudogenes were excluded from the gene list since pseudogenes are thought to be non-functional genes and used this ‘without pseudogene’ GSH atlas hereafter (FIG. 9A). Merging of the ATAC-seq atlas and GSH atlas resulted in the identification of 379 GSHs, which have at least one ATAC-seq peak as a part of the GSH or within 5 kb of the GSH boundaries, a region around an ATAC-seq peak which would likely be maintained open. These 379 GSHs were then ordered by average ATAC-seq signal intensity at the summit of the associated ATAC-seq peak across all seven samples (FIGS. 9A and 9B).

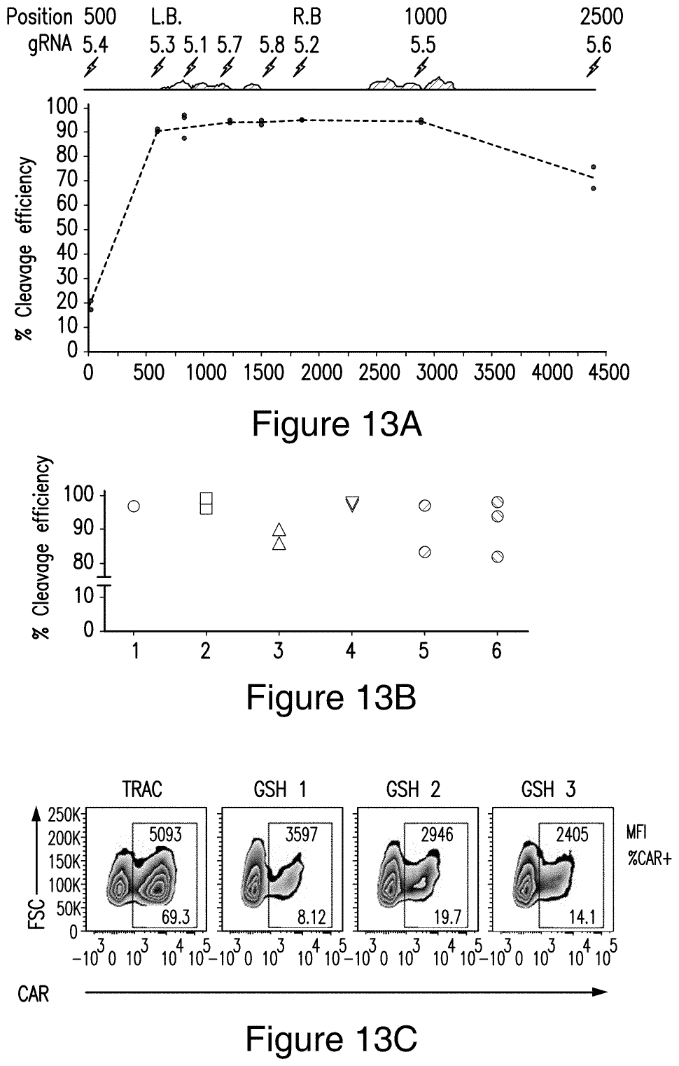

The 6 most accessible GSHs were then selected to test their cleavage efficiency. Four gRNAs per site were designed at the summit of the peak for all 6 GSHs such that all gRNAs possessed a Doench score>/=50 and specificity score>0.2 (Doench, J. G. et al. Nat. Biotechnol. 34, 1-12 (2016); Perez, A. R. et al. Nat. Biotechnol. 35, 347-349 (2017)). Electroporation of Cas9 mRNA and chemically modified sgRNAs (Hendel, A. et al. Nat. Biotechnol. 33, 985-989 (2015)) resulted in >90% cleavage efficiencies at all six GSHs tested at day 3 after electroporation (FIG. 9C). These high editing efficiencies supported that association of an GSH with a high intensity ATAC-seq peak affords a higher cleavage efficiency. However, it was still unclear if targeting only at the ATAC-seq peak summit would afford a high cleavage efficiency or whether targeting anywhere within the peak or even at a certain distance away from the peak could afford the same degree of cleavage. To test this, two GSHs were randomly chosen among the top 6, GSHs 1 and 5, to design gRNAs throughout the width of the peak and at specific distances away from the peak edges (up to 2.5 kb away) and analyzed the cleavage efficiencies at these sites in T cells from two independent donors (FIG. 9D and FIG. 13A). Although cleavage efficiency dropped slightly at one site at a distance of 2.5 kb away from one edge (FIG. 13A), high efficiency was generally maintained anywhere within the peak and at least up to about 500 bp away from the peak's edges.

Two gRNAs per GSH at the peak summit were further tested for four GSHs that had low ATAC-seq peak signal intensities and 3 GSHs identified previously (Papapetrou, E. P. et al. Nat. Biotechnol. 29, 73-78 (2011)) that had no associated ATAC-seq peaks. A multiple target site specific (MTSS) gRNA32 that targets 9 different loci which have different associated ATAC-seq peak signal intensities (FIGS. 12B and 12C) was additionally included. These controls further corroborated that an extragenic site with an associated high signal intensity ATAC-seq peak had a higher probability of efficient cleavage.

Expression of GSH-Encoded CAR and In Vitro Function

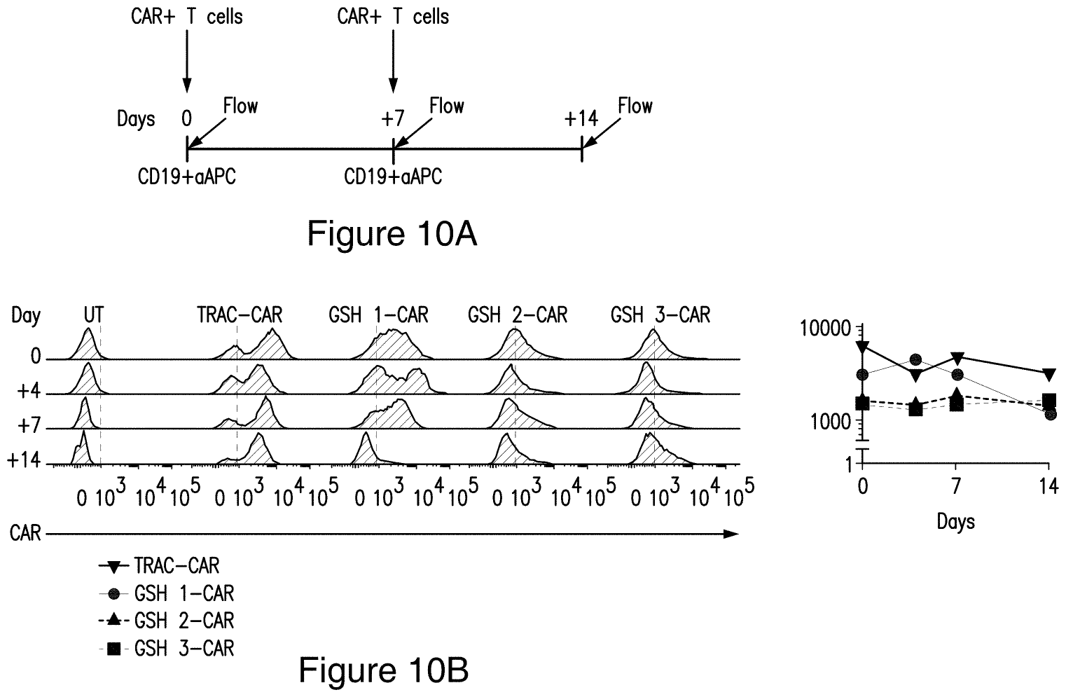

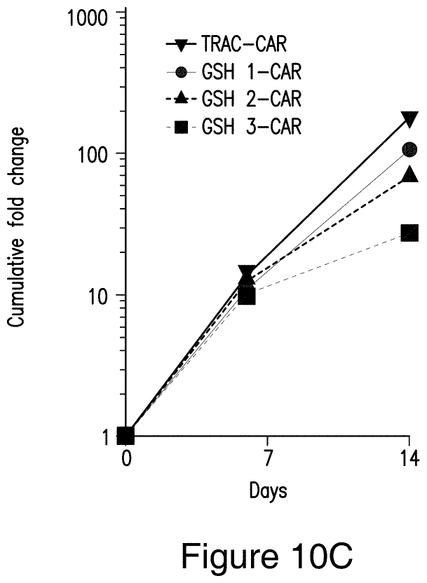

rAAV6 vectors were first designed encoding the 1928ζ-1xx CAR (Feucht, J. et al. et al. Nat. Med. 25, 82-88 (2018)) driven by the EF1α promoter (Eyquem, J., Poirot, L., Galetto, R., Scharenberg, A. M. & Smith, J. Biotechnol. Bioeng. (2013)) flanked by homology arms initially for GSHs-1, 2 and 3 (FIGS. 6 and 7). CAR targeting to the TRAC locus served as control and gold standard (Eyquem, J. et al. Nature 543, 113-117 (2017)). Expression of CAR was highest at GSH-1 among the GSHs, followed by GSH-2 and GSH-3, but lower than at TRAC (FIG. 13C). Commensurate with the CAR expression, GSH-1-CAR T cells displayed the highest cytolytic activity against CD19+ NALM6 leukemia cells equivalent to TRAC-CAR T cells, while GSH-2 and GSH-3 CAR-T cells showed reduced killing (FIG. 9E). To further analyze the functional capacity of the CAR-T cells and the effect of CAR expression on T-cell function, the proliferation of the CAR-T cells was measured over two weeks upon repeated encounter with antigen and examined cell-surface CAR expression at regular time intervals during this antigenic stimulation (FIGS. 10A and 10B). An upregulation of CAR expression was observed on the surface of GSH-1 CAR-T cells after the first exposure to antigen, reaching similar levels to CAR expression in TRAC targeted cells while the expression at GSH-2 and GSH-3 remained unchanged. This similarity in CAR expression levels at TRAC and GSH-1 upon antigen exposure explains the comparable cytolytic activity of these CAR-T cells (FIG. 9E). Upon the second exposure however, GSH-1 CAR-T cells failed to show the same level of CAR upregulation. This indicated that CAR expression silenced over time at all three GSHs, more rapidly at GSH-2 and 3 and gradually at GSH-1. The proliferation capacity of the GSH-CAR-T cells was lower in comparison to TRAC-CAR-T cells and was proportional to the CAR expression levels during the first week after transduction (FIG. 10C).

Characterization of GSHs and Association with Function

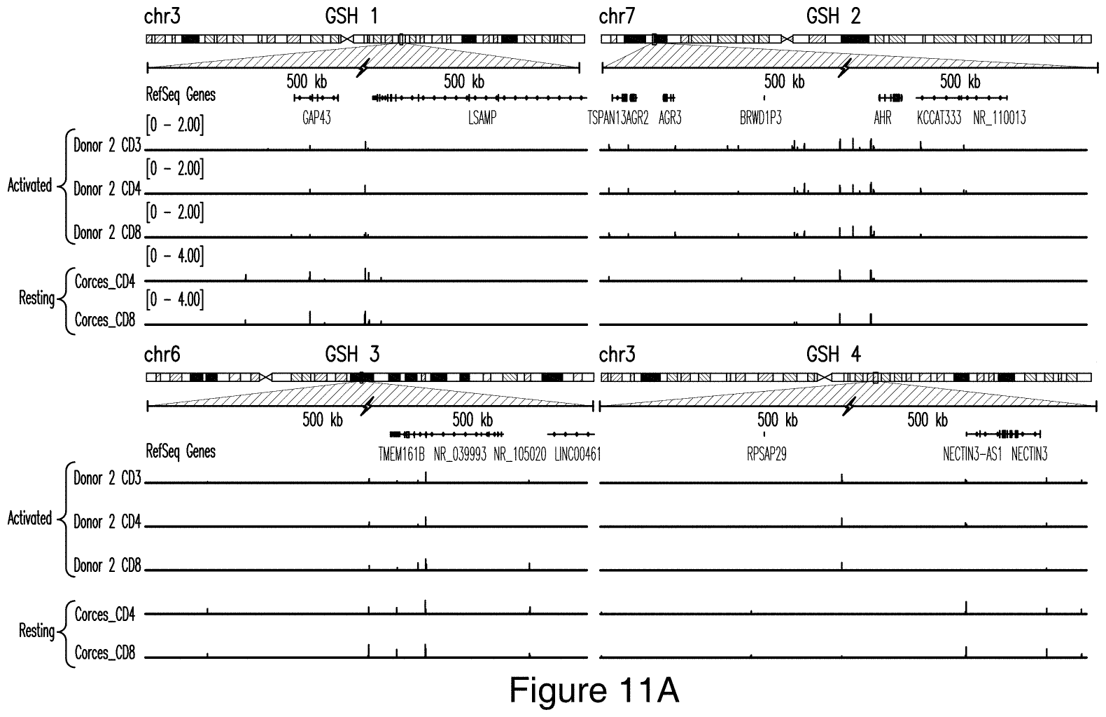

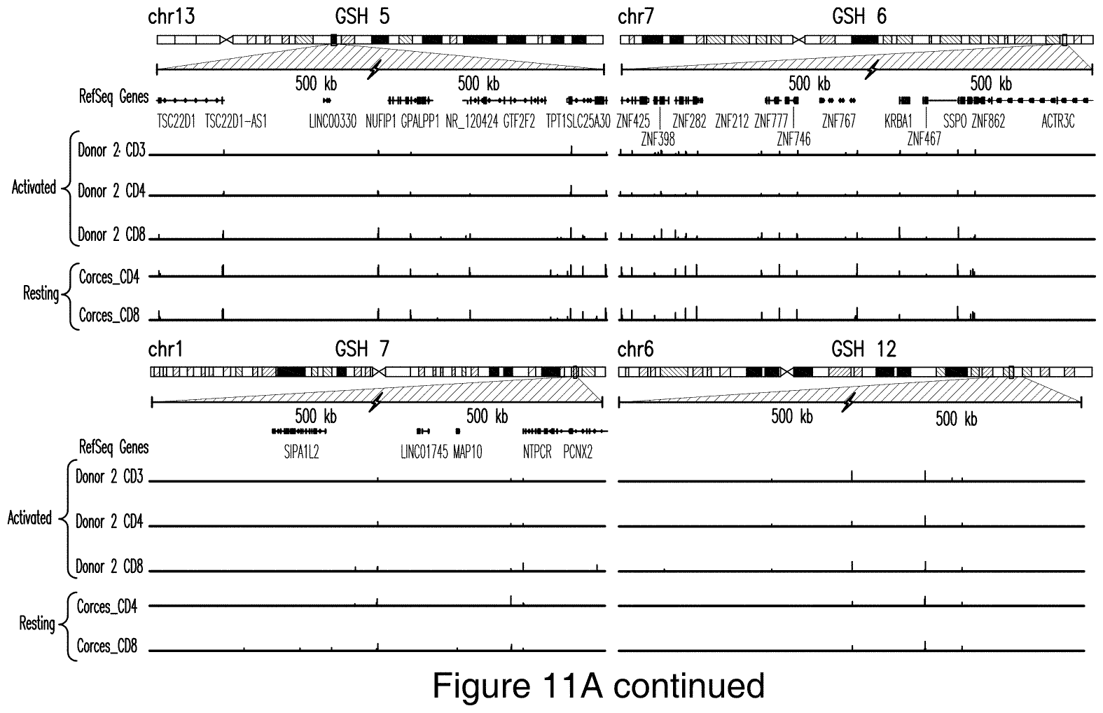

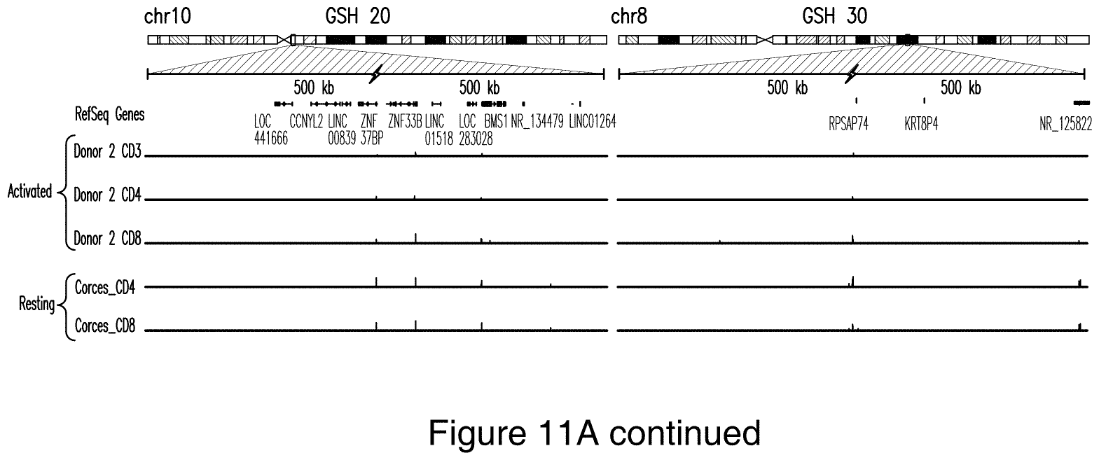

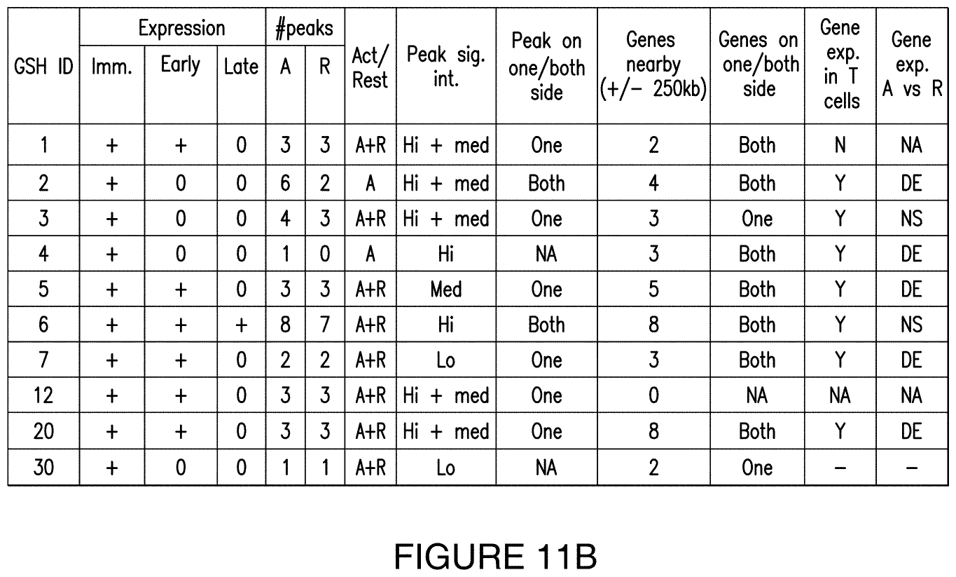

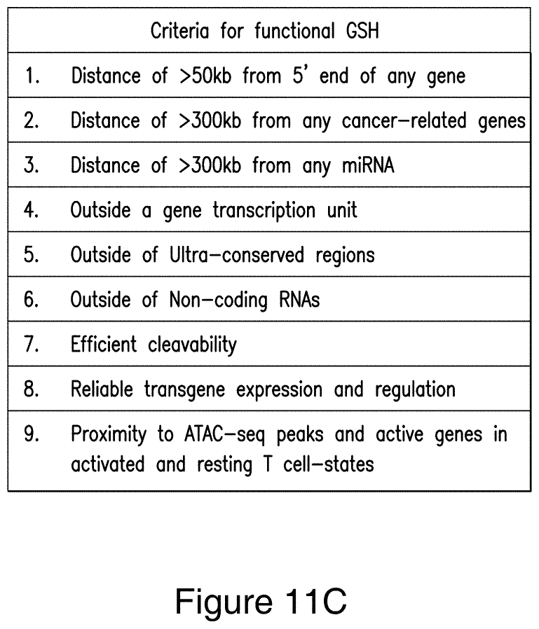

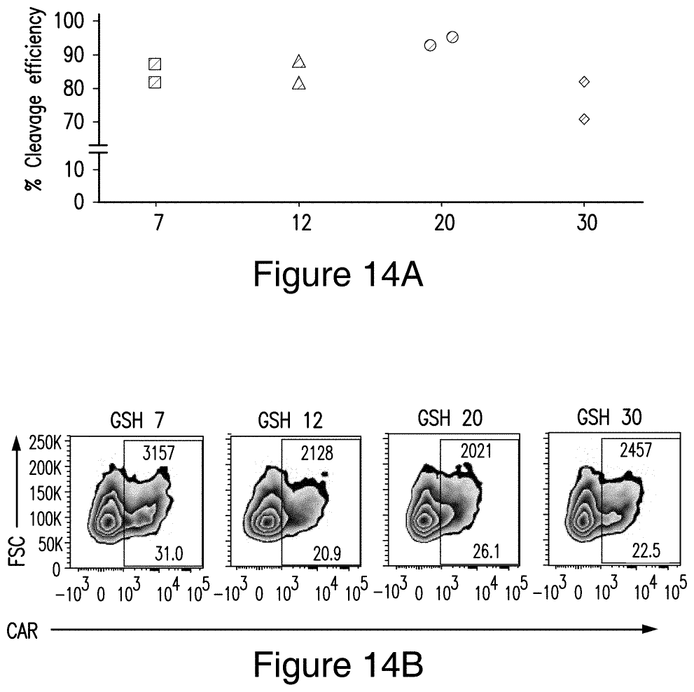

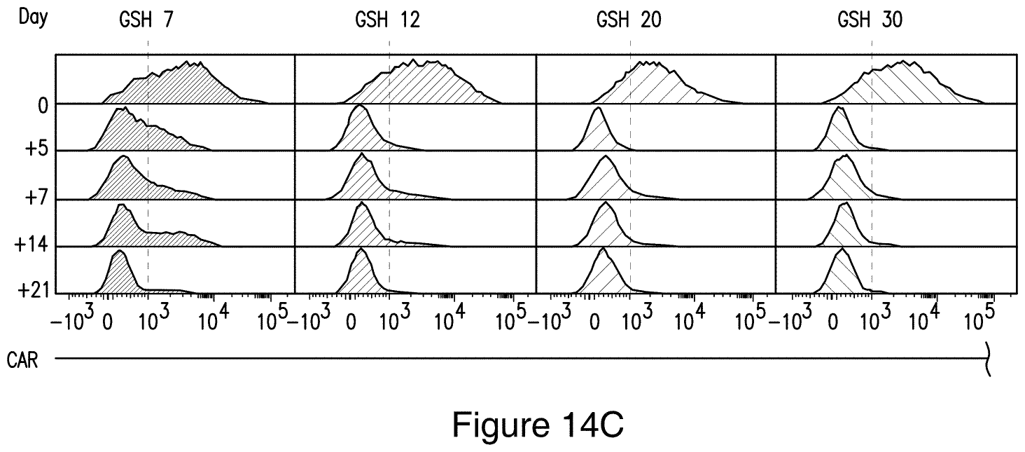

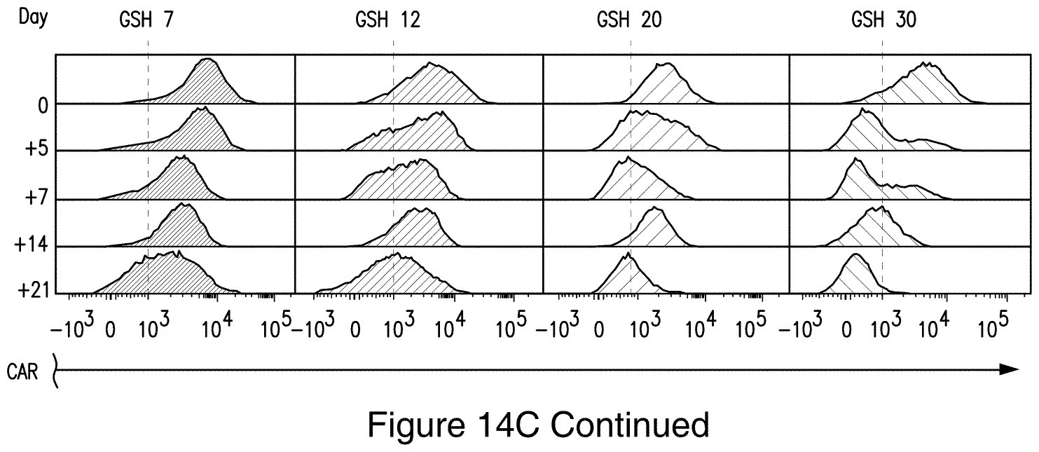

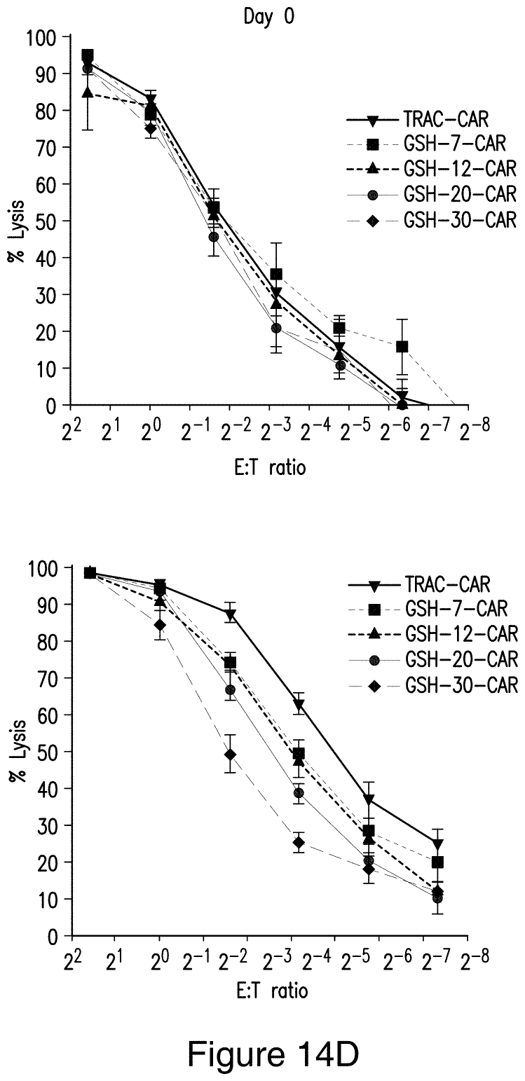

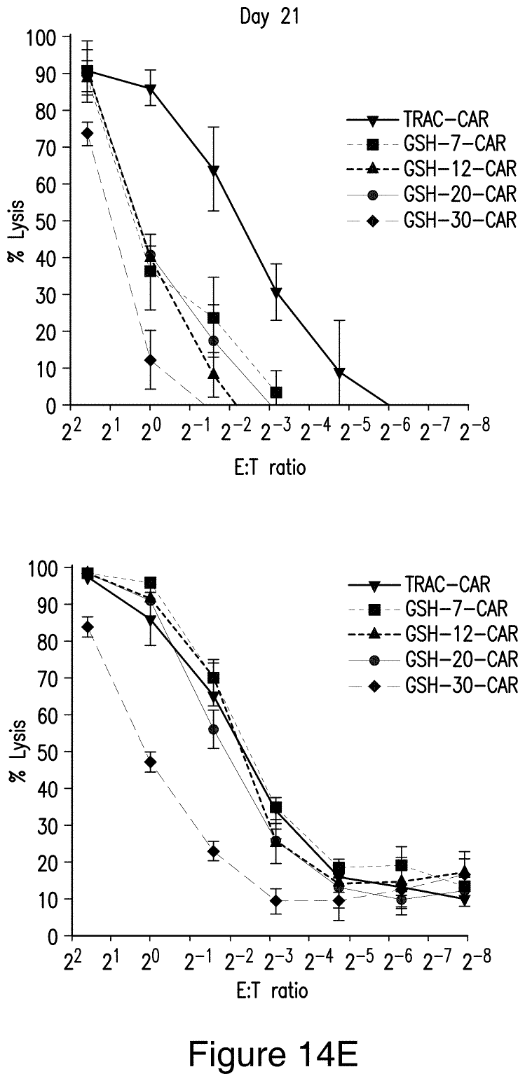

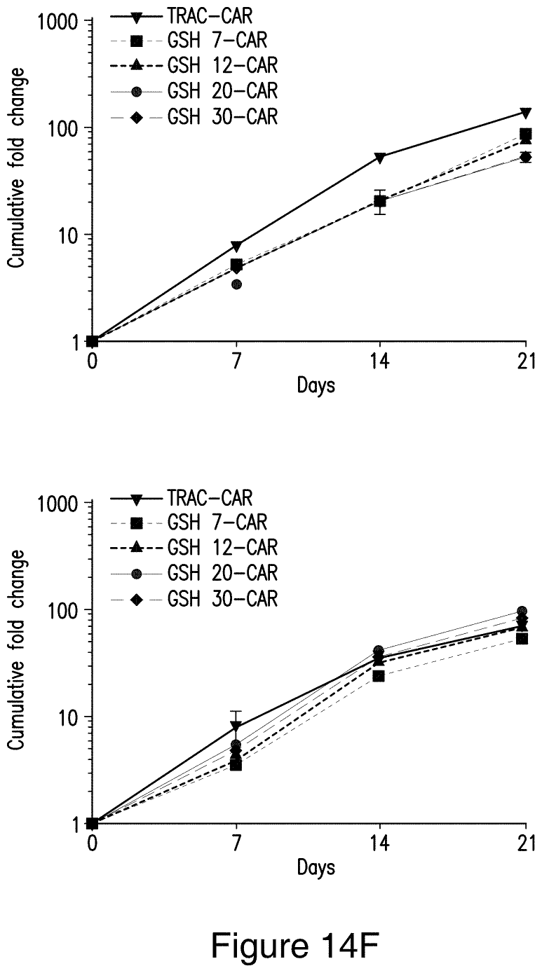

Given the widely different functional capacity of the CAR when integrated at different GSHs, it was sought to further understand the characteristics of an GSH with respect to its surrounding chromatin environment that dictate its functionality in the context of a T cell. This would help identify better functioning GSHs and these characteristics could then be integrated as part of the initial screening for GSHs. The reason for failure for most GSHs was inability or limited ability of expression upon activation which pointed to the inability of the locus to be held open in the resting state. Hence, the ATAC-seq data were analyzed at and around each of the six GSHs closely in activated and resting T cells. The activated T cell data used was the ATAC-seq data that were generated while the resting state data was obtained from Corces et al. (Corces, M. R. et al. Nat. Genet. 48, 1193-1203 (2016)). The expression of genes surrounding each of these sites in the resting and activated states was also studied. FIGS. 11A and 11B illustrate this information for all six GSHs. The best site associated with the best CAR-T cell function, GSH-6, was located within a pseudogene. To test whether the presence of the pseudogene alone granted better functionality to the GSH, 4 additional sites were tested where two of them (GSH 20 and 30) were located within a pseudogene or had a pseudogene very close to the site. The other two sites (GSH 7 and 12) were similar to intermediate (GSH 1-like) and poor performing (GSH 4-like) GSHs respectively in terms of presence of genes and ATAC-seq peaks around the sites (FIG. 11A). All 4 sites had a high intensity ATAC-seq peak and hence having a high cleavage efficiency. The cleavage efficiency, CAR integration, proliferation, expression and cytotoxicity at all these GSHs (FIGS. 14A-14F) were tested. All four GSHs showed high cleavage efficiencies with two gRNAs targeted at the summit of the peak and moderate initial CAR expression levels. Surprisingly, GSH 20 which seemed most similar to GSH 6 in terms of presence of genes around the site failed to perform as well as GSH 6 over the course of the multiple stimulations. The CTLs performed at day 0 and day 21 indicated that GSHs 7, 12 and 20 seemed to be in the intermediate performing GSH group whereas GSH 30 was the poorest performing GSH, similar to GSHs 2, 3 and 4 (FIGS. 14A-14F). This data indicated that the presence of a pseudogene at the site alone is not enough to grant better functionality to the GSHs. A closer examination of all GSHs, taking all data into consideration including the number of ATAC-seq peaks and genes within 250 kb around the site, respective gene expression and ATAC-seq peak signal intensity in the activated vs resting state (Tabulated in FIG. 11B) indicated that the following factors are responsible for deciding the activity of a particular GSH: 1) Proximity of peaks on either side of targeted peak 2) Intensity of proximal peaks 3) Presence of proximal and targeted peak in resting as well as activated state and 4) Presence of and expression of surrounding genes in resting and activated state. GSH 6 is characterized by the presence of high intensity ATAC-seq peaks as well as active genes in its proximity in the resting as well as activated state. These characteristics thus most likely influence the superior activity of GSH 6 over all other GSHs tested.

A number of future advances in human cell engineering based on gene addition depends on identifying safe genomic sites that afford dependable transgene expression. To achieve this goal, one may elect to target specific loci that provide desirable transgene regulation, e.g. the TRAC locus to express CARs (Eyquem, J. et al. Nature 543, 113-117 (2017)), or extragenic sites, the targeting of which does not entail disrupting an endogenous gene or known regulatory elements and may eventually accommodate large inserts encoding multiple genes. Criteria were previously proposed for the identification of such sites (Table 5, criteria 1-5 and Irion, S. et al. Nat. Biotechnol. 25, 1477-1482 (2007)), based on extensive insertional mutagenesis data accumulated in a number of clinical trials utilizing γ-retroviral and lentiviral vectors (Ellis, J. Hum. Gene Ther. 16, 1241-1246 (2005); Dunbar, C. E. Ann. N. Y. Acad. Sci. 1044, 178-182 (2005); Stein, S. et al. Nat. Med. 16, 198-204 (2010); Kohn, D. B., Sadelain, M. & Glorioso, J. C. Nat. Rev. Cancer 3, 477-488 (2003); Hacein-Bey-Abina, S. et al. J Clin Invest 118, 3132-3142 (2008); Howe, S. J. et al. J. Clin. Invest. 118, 3143-50 (2008)) and were utilized to retrospectively identify safe random integrations in clonal populations (Papapetrou, E. P. et al. Nat. Biotechnol. 29, 73-78 (2011)). Adding criteria for exclusion of non-coding RNAs, nuclease accessibility and chromatin context (criteria 7, 8, 9 FIG. 11C), demonstrating presently the feasibility of prospectively selected genomic regions that support therapeutic transgene expression of a CAR, matching the efficacy afforded by the reference TRAC locus.

To ensure highly efficient access to candidate GSH, a new criterion of chromatin accessibility was introduced. Cas9 would efficiently bind and cleave candidate GSH presenting with high chromatin accessibility (peak signal intensity) as assessed by ATAC-seq. It was indeed found that all 10 peaks meeting this criterion of high ATAC-seq peak signal intensity were efficiently cleaved at the center of the peak. At a distance from the peaks, accessibility was more variable, sometimes remaining high but markedly decreasing in other instances. Overlaying the safety criteria (1-6) with this one (7) reduced the number of candidate peaks in human primary T cells to 379.

The ATAC-seq profile of the different GSHs provides some insights into what may constitute a more favorable site for sustained expression in T cells. The surrounding ATAC-seq peaks and gene expression profiles in resting and activated T cells differed slightly between the 10 GSHs where the CAR cDNA was integrated. Proximity to genes—while complying with the GSH criteria—that are active in both resting and activated T cell states and presence of ATAC-seq peaks in both states was observed at GSH6. These features were not all found at the other GSHs. These may thus represent a screening criterion to add to the presently disclosed GSH requirements for optimal T cell genome editing (FIG. 11C).

### Methods

Generation of GSH Atlas.

The first six criteria for GSHs (Table 5) were applied to build a Genomic safe harbor atlas (GSH) atlas based on the Human GRCh37/hg19 assembly. Gene annotation information for criteria 1 and 4 were obtained from GENCODE version 25 and RefSeq_NM database from NCBI. Data for cancer-related genes were obtained by combining oncogene lists from Bushman group allOnco list (v2) (http://www.bushmanlab.org/links/genelists), COSMIC Cancer gene census v78 (https://cancer.sanger.ac.uk/cosmic) and CancerGeneticsWeb (http://www.cancer-genetics.org/). miRNA data was obtained from hg19 sno/miRNA track in UCSC Genome Browser and GENCODE entries for miRNAs. The data for UCRs in the human genome was obtained from http://users.soe.ucsc.edu/˜jill/ultra.html (Bejerano, G. et al. Science 304, 1321-1326 (2004)). As the genomic coordinates used in the publication were from an older assembly, the coordinates were converted to hg19 using UCSC lift genome annotations tool. Data for Non-coding RNA (ncRNA) list was obtained from NONCODE v5 (www.noncode.org) and GENCODE ncRNA entries. Pseudogene annotation from GENCODE was used to either include or exclude pseudogenes from the gene list to create two atlases—With pseudogenes and Without pseudogenes. The assembly gaps as mentioned on the UCSC Genome Browser for hg19 genome were excluded.

ATAC-Seq Atlas for Human T Cell Genome.

Peripheral blood mononuclear cells were obtained by density gradient centrifugation from peripheral blood of three healthy adult human volunteers. T cells were purified using the Pan T Cell Isolation Kit (Miltenyi Biotec) and stimulated with CD3/CD28 T cell Activator Dynabeads (Invitrogen) (1:1 beads:cell) and cultured in X-VIVO 15 Serum-free Hematopoietic Cell Medium (Lonza), supplemented with 5% human serum (Gemini Bio-Products) and 200 U ml⁻¹ IL-2 (Miltenyi Biotec). Cells were cultured at 10⁶ cells per ml. CD3/CD28 beads were magnetically removed 48 h after initiating T cell activation. At day 3 after isolation and activation, the T cells were sorted into CD4 and CD8 fractions from two donors by magnetic separation through negative selection using Human CD4-biotin and Human CD8-biotin beads (Miltenyi Biotec) and anti-biotin beads (Miltenyi Biotec). CD3, CD4 and CD8 cells from donors 2 and 3 and only CD3 cells from donor 1 were collected and 50,000 cells were frozen in freezing medium (10% DMSO in FBS) for ATAC-seq analysis. ATAC-seq was performed by the MSKCC IGO core. The method used for ATAC-seq was as described previously (Buenrostro, J. D., Wu, B., Chang, H. Y. & Greenleaf, W. J. Curr. Protoc. Mol. Biol. 109, 21.29.1-21.29.9 (2015)) but with the change that the transposition reaction was performed at 42° C. for 45 mins since this condition gave a better library prep. All ATAC libraries were sequenced using paired-end, dual-index sequencing on an Illumina HiSeq instrument with 2×50 bp reads for at least 30 million read pairs.

ATAC-Seq Data Processing.

Raw FASTQ reads were trimmed with Trimmomatic (Bolger, A. M., Lohse, M. & Usadel, B. Bioinformatics 30, 2114-2120 (2014)) and aligned to hg19 using Bowtie2 (Langmead, B. & Salzberg, S. L. Nat. Methods 9, 357-359 (2012)). Bam files were filtered based on map quality and PE concordance, duplicated reads were removed and tn5 specific read shift was performed. To call peaks, data were aggregated by cell type and peak calling was performed using MACS2 (Zhang, Y. et al. Genome Biol. 9, R137 (2008)) and filtered using ENCODE hg19 blacklist (Amemiya, H. M., Kundaje, A. & Boyle, A. P. Sci. Rep. 9, 9354 (2019)). Irreproducible discovery rate (IDR) analysis was performed for all replicate pairs. Peaks with global IDR <0.05 were considered as reproducible peaks. The ATAC-seq data from the Corces et al. study available publicly was also processed similarly, visualized using the IGV genome browser by setting to the same signal range to view all of the GSH regions.

Identification of Candidate GSHs.

The Genomic Safe Harbor atlas (without pseudogenes) and the ATAC-seq atlas were overlaid to find GSHs associated with an ATAC-seq peak. 21,566 ATAC-seq peaks that are shared across all samples were overlapped with GSH atlas to identify 379 ATAC-seq peaks that had an GSH within 5 kb. These ATAC-seq peaks were termed as GSH peaks and were then ranked by the average signal intensity (RPM) at the summit to identify candidate GSHs for further testing.

Antigen Stimulation and In Vitro Proliferation Assays.

For use in weekly proliferation assay, 3 days after AAV6 transduction, CAR targeted cells were purified using magnetic Biotin-SP (long spacer) AffiniPure F(ab′)2 Fragment Goat Anti-Mouse IgG, F(ab′)2 Fragment Specific antibody (Jackson ImmunoResearch, 115-066-072), anti-biotin microbeads and MS columns (Miltenyi Biotec). The purified cells were cultured for 4 days as described before. NIH/3T3 cells expressing human CD19 were used as artificial antigen-presenting cells (AAPCs). For weekly stimulations, 3×10⁵ irradiated CD19+ AAPCs were plated in 24-well plates 12 h before the addition of 5×10⁵ CAR+ purified T cells in X-VIVO 15 medium containing 5% human serum, 5 ng ml⁻¹ IL7 and 5 ng ml⁻¹ IL15 (Peprotech). Every 2 days, cells were counted and media was added to reach a concentration of 2×10⁶ cells per ml. For each condition, T cells were analyzed by flow cytometry for CAR expression at time points mentioned in the respective figures. The antibody used for CAR staining was Alexa Fluor 647 AffiniPure F(ab′)2 Fragment Goat Anti-Mouse IgG, F(ab′)2 Fragment Specific (Jackson ImmunoResearch, 115-606-072). For keeping the CAR MFI comparable across all experiments and time-points, Rainbow Fluorescent Particles (BD Biosciences, 556298) were used.

Luciferase Based Cytotoxicity Assays.

NALM6-expressing CD19-FFLuc-GFP served as target cells. The effector CAR+ T cells and target cells were cocultured in triplicates at the indicated effector/target ratio using black-walled 96-well plates with 15000 target cells in a total volume of 100 μl per well in NALM6 medium. Target cells alone were plated at the same cell density to determine the maximal luciferase expression (relative light units (RLU)); 18 h later, 100 μl luciferase substrate (Bright-Glo; Promega) was directly added to each well. Emitted light was detected in a luminescence plate reader (TECAN Spark Reader). Lysis was determined as (1−(RLUsample)/(RLUmax))×100.

Mouse cell depletion kit (Miltenyi Biotec) was used for mouse cell depletion from bone marrow according to manufacturer's instructions and flow-through cells were then used for the ex-vivo co-culture and cytotoxicity assay with NALM6 cells as described above.

Antibodies and Staining for Flow Cytometry.

The following fluorophore-conjugated antibodies were used. From BD Biosciences: APC-Cy7 mouse anti-human CD8; BUV395 mouse anti-human CD4; PE-Cy7 mouse anti-human CD4; BV421 mouse anti-human CD62L; BV650 mouse anti-human CD45RA; BV510 mouse anti-human CD279 (PD-1); BUV737 mouse anti-human CD19. From BioLegend: PE mouse anti-human CD45; BV785 mouse anti-human TIM3 (CD366); BV421 mouse anti-human CD19. From eBioscience: PerCP-eFluor 710 CD223 (LAG-3) Monoclonal Antibody (3DS223H). 7-AAD (BD Biosciences) and DAPI solution (BD Biosciences) were used as viability dyes. For CAR staining, an Alexa Fluor 647 AffiniPure F(ab′)2 Fragment Goat Anti-Mouse IgG, F(ab′)2 fragment specific antibody was used (Jackson ImmunoResearch). For cell counting, CountBright Absolute Counting Beads were added (Invitrogen) according to the manufacturer's instructions. For in vivo experiments, Normal mouse serum (EMD Millipore) and FcR Blocking Reagent, mouse (Miltenyi Biotec) were used to block mouse Fc receptors.

Flow cytometry was performed on an LSRII or LSRFortessa instrument (BD Biosciences). Data were analyzed with the FlowJo software v.10.1 (FlowJo LLC).

Statistical Analysis.

All statistical analyses were performed using the Prism 7 (GraphPad) software. No statistical methods were used to predetermine sample size. Statistical comparisons between two groups were determined by two-tailed parametric or nonparametric (Mann-Whitney U-test) t-tests for unpaired data or by two-way Anova for multiple comparisons. For in-vivo experiments, the overall survival was depicted by a Kaplan-Meier curve. P values<0.05 were considered to be statistically significant. The statistical test used for each figure is described in the corresponding figure legend.

Although the presently disclosed subject matter and certain of its advantages have been described in detail, it should be understood that various changes, substitutions and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method for identifying a genomic safe harbor (GSH), comprising: (i) screening a plurality of loci within a genome, (ii) evaluating the position of the loci, and (iii) identifying a locus as an GSH if the locus is:
(a) located at a distance of more than about 50 kb from the 5′ end of each gene of the genome;
(b) located at a distance of more than about 300 kb from each cancer-related gene of the genome;
(c) located outside each gene transcription unit of the genome;
(d) located outside of each ultra-conserved region of the genome;
(e) located outside of each non-coding RNA region of the genome; and
(f) located at a distance more than about 300 kb from each microRNA (miRNA) gene of the genome.

2. The method of claim 1, further comprising (iv) measuring cleavage efficiency of a gene editing system that is delivered at the loci and selecting a locus as an GSH if the cleavage efficiency of the gene editing system at the locus is at least about 90%.

3. The method of claim 2, further comprising selecting a locus as an GSH if the cleavage efficiency of the gene editing system at the locus is at least about 95%.

4. The method of claim 3, wherein the gene editing system is a CRISPR gene editing system.

5. The method of claim 1, further comprising (v) measuring expression of a transgene that is integrated at the loci, and selecting a locus as an GSH if the transgene integrated at the locus is expressed at a detectable level.

6. The method of claim 5, wherein the transgene encodes a molecule.

7. The method of claim 6, wherein the molecule is an antigen-recognizing receptor that binds to an antigen.

8. The method of claim 7, wherein the antigen-recognizing receptor is selected from the group consisting of a chimeric antigen receptor (CAR), a T-cell receptor (TCR), a chimeric co-stimulating receptor (CCR), and a TCR like fusion molecule.

9. The method of claim 7, wherein the antigen-recognizing receptor is a chimeric antigen receptor (CAR).

10. The method of claim 8, further comprising measuring the expression of the CAR about four (4) days, about one (1) week, or about two (2) weeks from initial stimulation of the antigen

11. The method of claim 1, further comprising (vi) determining whether the loci comprise a pseudogene, and selecting a locus as an GSH if the locus comprises a pseudogene.

12. The method of claim 1, further comprising (vii) determining the chromatin accessibility of the loci across the genome, and selecting a locus as an GSH if the locus has higher chromatin accessibility than about 90% of the plurality of loci screened.

13. The method of claim 12, wherein the chromatin accessibility is determined by an Assay for Transposase-Accessible Chromatin with high-throughput sequencing (ATAC-seq).

14. The method of claim 13, further comprising selecting a locus as an GSH if the locus is located at a distance of about 5 kb from an ATAC-seq peak.

15. The method of claim 14, wherein the ATAC-seq peak is present in both resting and activated states of a cell.

16. The method of claim 12, further comprising selecting a locus as an GSH if the locus is located at a distance of up to about 250 kb from at least one gene that is activated and expressed in both resting and activated states of a cell.

17. The method of claim 12, further comprising selecting a locus as an GSH if ATAC-seq peaks are present on both sides of the locus.

18. The method of claim 17, wherein the ATAC-seq peaks is located at a distance of up to about 250 kb from the locus.

19. The method of claim 17, wherein the ATAC-seq peaks are present in both resting and activated states of a cell.

20. The method of claim 15, wherein the cell is a T cell.
